(19) United States
(12) Patent Application Publication
(10) Pub. No.: US 2008/0118910 A1
(43) Pub. Date: May 22, 2008

(54) CONTROL NUCLEIC ACID CONSTRUCTS FOR USE WITH GENOMIC ARRAYS

(76) Inventor: Stephen B. Milligan, Los Altos, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/469,388

(22) Filed: Aug. 31, 2006

(51) Int. Cl. C12Q 1/70; C12Q 1/68; C07H 21/04

(52) U.S. Cl. 435/5; 435/6; 536/24.1

(57) ABSTRACT

In some embodiments, nucleic acid constructs are provided which comprise a nucleic acid vector having an insert comprising a control nucleic acid molecule. The control nucleic acid molecule comprises a sequence complementary to a negative control probe in a microarray. Methods and kits for using the nucleic acid constructs as spiking reagents in microarray analysis are disclosed.

### BACKGROUND

Chemical arrays have gained prominence in biological research and serve as valuable diagnostic tools in the healthcare industry. A fundamental principle upon which array assays are based is that of specific recognition. Probe molecules affixed to the array can specifically recognize and bind target molecules in a sample, either by sequence-mediated binding affinities, binding affinities based on conformational or topological properties of probe and target molecules, or binding affinities based on spatial distribution of electrical charge on the surfaces of target and probe molecules.

An array generally includes a substrate upon which a regular pattern of features is prepared by various manufacturing processes. The array typically has a grid-like two-dimensional pattern of features. For nucleic acid arrays, each feature of the array contains a large number of oligonucleotides covalently bound to the surface of the feature. These bound oligonucleotides are known as probes. In general, chemically distinct probes are bound to the different features of an array, so that each feature corresponds to a particular known nucleotide sequence.

Once an array has been prepared, the array can be exposed to a sample solution containing target molecules (such as DNA or RNA) labeled with fluorophores, chemiluminescent compounds, or radioactive atoms. The labeled target molecules then hybridize to the complementary probe molecules on the surface of the array. Targets, such as labeled DNA molecules that are not complementary to any of the probes bound to array surface do not hybridize as readily and tend to remain in solution. The sample solution is then rinsed from the surface of the array, washing away any unbound labeled molecules. Finally, the bound labeled molecules are detected via optical or radiometric scanning.

Scanning of an array by an optical scanning device or radiometric scanning device generally produces a scanned image comprising a plurality of pixels corresponding to features on the array, with each pixel having a corresponding signal intensity. Typically, an array-data-processing program then manipulates these signal intensities and produces experimental or diagnostic results.

There is a need for exogenous nucleic acid controls (“spikes”) for microarray analysis. Variations in sample preparation, hybridization conditions, and array quality can influence the values determined for the copy number levels of different samples. Constructing large databases of samples prepared differently and hybridized to different array types can be especially challenging. The use of quality-assured control polynucleotides during sample preparation and during hybridization to microarrays greatly enhances the ability to normalize data and to compare experiments, as well as to monitor each step of the assay.

### SUMMARY

In some aspects, a nucleic acid construct, useful as a spiking reagent in microarray analysis, is provided. In some embodiments, a nucleic acid construction comprises a nucleic acid vector comprising an insert comprising a sequence complementary to a negative control sequence. The vector can be a viral nucleic acid vector, a non-limiting example of which is lambda phage gt11. In some embodiments, a double stranded control nucleic acid is inserted into a restriction site (such as, for example, an EcoR1 restriction site) in the vector. In some embodiments, a PCR amplification product of the nucleic acid construct, which includes the insert, is obtained, and can be used as a spiking reagent. The length of the nucleic acid construct can range in size from about 1 kilobases (kb) to about 100 kb. The length of the insert can be in the range of about 10 to about 500 bases. In some embodiments, the insert has a length of 60 bases. Also provided, are collections of different nucleic acid constructs, wherein each different insert in the collection has a different sequence. The collection can comprise defined sequences present in known ratios. Nucleic acid constructs in a collection can span a range of concentrations, such as 2, 3, 4, 5, 6, 7, 8, or more logs of magnitude.

Provided herein are methods for preparing a control nucleic acid construct as described herein.

In some embodiments, the nucleic acid constructs are designed to simulate genomic DNA. The length of a construct can be about 10% to 200%, about 50% to about 150% or about 100% of the length of DNA fragments in a sample being analyzed.

In some aspects, there are provided methods for use in the preparation of a nucleic acid sample for microarray analysis. The methods can comprise the steps of: adding a nucleic acid construct, as described herein, to a sample, and subjecting said sample to a plurality of processing steps. The processing steps can include a fragmentation process. Examples of such microarray analyses include a CGH assay or a location analysis assay.

In some aspects, there are provided methods for monitoring hybridization of an eukaryotic nucleic acid sample to a nucleic acid array. The method can comprise the steps of: (a) providing a nucleic acid array comprising a negative control probe and a plurality of nucleic acid test probes that specifically bind to eukaryotic nucleic acid targets; (b) providing a spiked sample comprising a eukaryotic nucleic acid sample and a nucleic acid construct, the nucleic acid construct comprising an insert that specifically binds to the negative control probe; (c) fragmenting the spiked sample; (d) contacting the spiked sample with the array; and (e) determining whether hybridization occurred between the negative control probe and the control nucleic acid molecule.

In some aspects, provided herein are kits comprising a nucleic acid construct as described herein.

The nucleic acid constructs can be added to a sample of target nucleic acids being analyzed to allow a user to assess any degradation in the overall performance of the microarray (including, but not limited to, signal-to-noise, dynamic range, linearity of response, and background).

### BRIEF DESCRIPTION OF THE DRAWINGS

Embodiments can be more completely understood in connection with the following drawings, in which:

FIG. 1 schematically illustrates some embodiments of a control nucleic acid construct.

FIG. 2 illustrates data obtained from a hybridization assay.

FIG. 3 illustrates a schematic diagram of a system for manufacturing arrays.

FIG. 4 illustrates an example of a general purpose computing system.

FIG. 5 shows operations performed in some embodiments.

FIG. 6 shows operations of similarity screening performed in some embodiments.

### DETAILED DESCRIPTION

Before describing the present disclosure in detail, it is to be understood that this disclosure is not limited to specific compositions, method steps, or equipment, as such can vary. It is also to be understood that the terminology used herein is for the purpose of describing particular embodiments only, and is not intended to be limiting. Methods recited herein can be carried out in any order of the recited events that is logically possible, as well as the recited order of events. Furthermore, where a range of values is provided, it is understood that every intervening value, between the upper and lower limit of that range and any other stated or intervening value in that stated range is encompassed within the present disclosure. Also, it is contemplated that any optional feature of the inventive variations described can be set forth and claimed independently, or in combination with any one or more of the features described herein.

Unless defined otherwise below, all technical and scientific terms used herein have the same meaning as commonly understood by one of ordinary skill in the art to which this disclosure belongs. Still, certain elements are defined herein for the sake of clarity.

All literature and similar materials cited in this application, including but not limited to patents, patent applications, articles, books, treatises, and internet web pages, regardless of the format of such literature and similar materials, are expressly incorporated by reference in their entirety for any purpose. In the event that one or more of the incorporated literature and similar materials differs from or contradicts this application, including but not limited to defined terms, term usage, described techniques, or the like, this application controls.

The publications discussed herein are provided solely for their disclosure prior to the filing date of the present application. Nothing herein is to be construed as an admission that the present disclosure is not entitled to antedate such publication by virtue of prior invention. Further, the dates of publication provided can be different from the actual publication dates, which need to be independently confirmed.

It must be noted that, as used in this specification and the appended claims, the singular forms “a”, “an” and “the” include plural referents unless the context clearly dictates otherwise. Thus, for example, reference to “a biopolymer” can include more than one biopolymer.

### Definitions

The following definitions are provided for specific terms that are used in the following written description.

A “biopolymer” is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. As such, this term includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. Specifically, a “biopolymer” includes deoxyribonucleic acid or DNA (including cDNA), ribonucleic acid or RNA and oligonucleotides, regardless of the source.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “mRNA” means messenger RNA.

A “biomonomer” references a single unit, which can be linked with the same or other biomonomers to form a biopolymer (for example, a single amino acid or nucleotide with two linking groups one or both of which can have removable protecting groups). A biomonomer fluid or biopolymer fluid reference a liquid containing either a biomonomer or biopolymer, respectively (typically in solution).

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. Nucleotide sub-units of deoxyribonucleic acids are deoxyribonucleotides, and nucleotide sub-units of ribonucleic acids are ribonucleotides.

An “oligonucleotide” generally refers to a nucleotide multimer of about 10 to 100 nucleotides in length, while a “polynucleotide” or “nucleic acid” includes a nucleotide multimer having any number of nucleotides.

The term “base composition properties” shall refer to properties of a sequence related to base composition. By way of example, while not limiting the term, base composition properties can include the percentage of A, C, T, and G sequences within a given probe sequence.

The term “primary structural features” as used herein shall refer to structural features of a sequence related the contiguous positioning of bases in the sequence. While not limiting the term, an example of a primary structural feature is a homopolymeric run.

The term “homopolymeric run” as used herein shall refer to a portion of a base sequence wherein a given base is repeated more than once. By way of example, a sequence contains the contiguous bases “TTTTT” would be considered to have a homopolymeric run.

The term “secondary structural features” as used herein shall refer to structural features (predicted or empirical) of a sequence caused by the interaction between both contiguous and non-contiguous bases in the sequence. While not limiting the term, an example of a secondary structural feature is a hairpin loop structure.

As used herein, the term “thermodynamic characteristics” shall refer to characteristics of a sequence described in thermodynamic terms. By way of example, while not limiting the term, thermodynamic characteristics of a given sequence can include the Gibbs free energy of hybridization of that sequence with another sequence. As a further example, while not limiting the term, thermodynamic characteristics of a given sequence can include the melting temperature (Tm) of the sequence.

A chemical “array”, unless a contrary intention appears, includes any one, two or three-dimensional arrangement of addressable regions bearing a particular chemical moiety or moieties (for example, biopolymers such as polynucleotide sequences) associated with that region, where the chemical moiety or moieties are immobilized on the surface in that region. By “immobilized” is meant that the moiety or moieties are stably associated with the substrate surface in the region, such that they do not separate from the region under conditions of using the array, e.g., hybridization and washing and stripping conditions. As is known in the art, the moiety or moieties can be covalently or non-covalently bound to the surface in the region. For example, each region can extend into a third dimension in the case where the substrate is porous while not having any substantial third dimension measurement (thickness) in the case where the substrate is non-porous. An array can contain more than ten, more than one hundred, more than one thousand more than ten thousand features, or even more than one hundred thousand features, in an area of less than 20 cm² or even less than 10 cm². For example, features can have widths (that is, diameter, for a round spot) in the range of from about 10 μm to about 1.0 cm. In other embodiments each feature can have a width in the range of about 1.0 μm to about 1.0 mm, such as from about 5.0 μm to about 500 μm, and including from about 10 μm to about 200 μm. Non-round features can have area ranges equivalent to that of circular features with the foregoing width (diameter) ranges. A given feature is made up of chemical moieties, e.g., nucleic acids, that bind to (e.g., hybridize to) the same target (e.g., target nucleic acid), such that a given feature corresponds to a particular target. At least some, or all, of the features are of different compositions (for example, when any repeats of each feature composition are excluded the remaining features can account for at least 5%, 10%, or 20% of the total number of features). Interfeature areas will typically (but not essentially) be present which do not carry any polynucleotide. Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations. An array is “addressable” in that it has multiple regions (sometimes referenced as “features” or “spots” of the array) of different moieties (for example, different polynucleotide sequences) such that a region at a particular predetermined location (an “address”) on the array will detect a particular target or class of targets (although a feature can incidentally detect non-targets of that feature). The target for which each feature is specific is, in representative embodiments, known. An array feature is generally homogenous in composition and concentration and the features can be separated by intervening spaces (although arrays without such separation can be fabricated).

The phrase “oligonucleotide bound to a surface of a solid support” or “probe bound to a solid support” or a “target bound to a solid support” refers to an oligonucleotide or mimetic thereof, e.g., PNA, LNA or UNA molecule that is immobilized on a surface of a solid substrate, where the substrate can have a variety of configurations, e.g., a sheet, bead, particle, slide, wafer, web, fiber, tube, capillary, microfluidic channel or reservoir, or other structure. In some embodiments, the collections of oligonucleotide elements employed herein are present on a surface of the same planar support, e.g., in the form of an array. It should be understood that the terms “probe” and “target” are relative terms and that a molecule considered as a probe in certain assays can function as a target in other assays.

“Addressable sets of probes” and analogous terms refer to the multiple known regions of different moieties of known characteristics (e.g., base sequence composition) supported by or intended to be supported by an array surface, such that each location is associated with a moiety of a known characteristic and such that properties of a target moiety can be determined based on the location on the array surface to which the target moiety binds under stringent conditions.

An “array layout” or “array characteristics”, refers to one or more physical, chemical or biological characteristics of the array, such as positioning of some or all the features within the array and on a substrate, one or more feature dimensions, or some indication of an identity or function (for example, chemical or biological) of a moiety at a given location, or how the array should be handled (for example, conditions under which the array is exposed to a sample, or array reading specifications or controls following sample exposure).

In some embodiments, an array is contacted with a nucleic acid sample under stringent assay conditions, i.e., conditions that are compatible with producing bound pairs of biopolymers of sufficient affinity to provide for the desired level of specificity in the assay while being less compatible to the formation of binding pairs between binding members of insufficient affinity. Stringent assay conditions are the summation or combination (totality) of both binding conditions and wash conditions for removing unbound molecules from the array.

As known in the art, “stringent hybridization conditions” and “stringent hybridization wash conditions” in the context of nucleic acid hybridization are sequence dependent, and are different under different experimental parameters. Stringent hybridization conditions include, but are not limited to, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization in 0.5 M NaHPO₄, 7% sodium dodecyl sulfate (SDS), 1 mM EDTA at 65° C., and washing in 0.1×SSC/0.1% SDS at 68° C. can be performed. Additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

Wash conditions used to remove unbound nucleic acids can include, e.g., a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature. Other methods of agitation can be used, e.g., shaking, spinning, and the like.

Stringent hybridization conditions can also include a “prehybridization” of aqueous phase nucleic acids with complexity-reducing nucleic acids to suppress repetitive sequences. For example, certain stringent hybridization conditions include, prior to any hybridization to surface-bound polynucleotides, hybridization with Cot-1 DNA, or the like.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and can also be employed, as appropriate. The term “highly stringent hybridization conditions” as used herein refers to conditions that are compatible to produce complexes between complementary binding members, i.e., between immobilized probes and complementary sample nucleic acids, but which do not result in any substantial complex formation between non-complementary nucleic acids (e.g., any complex formation which cannot be detected by normalizing against background signals to interfeature areas and/or control regions on the array).

Additional hybridization methods are described in references describing CGH techniques (Kallioniemi et al., Science 1992; 258:818-821 and WO 93/18186). Several guides to general techniques are available, e.g., Tijssen, Hybridization with Nucleic Acid Probes, Parts I and II (Elsevier, Amsterdam 1993). For descriptions of techniques suitable for in situ hybridizations see, Gall et al. Meth. Enzymol. 1981; 21:470-480 and Angerer et al., In Genetic Engineering: Principles and Methods, Setlow and Hollaender, Eds. Vol 7, pgs 43-65 (Plenum Press, New York 1985). See also U.S. Pat. Nos. 6,335,167; 6,197,501; 5,830,645; and 5,665,549; the disclosures of which are herein incorporated by reference.

In the case of an array, the “target” will be referenced as a moiety in a mobile phase (typically fluid), to be detected by probes (“target probes”) which are bound to the substrate at the various regions. However, either of the “target” or “target probes” may be the one which is to be detected by the other (thus, either one could be an unknown mixture of polynucleotides to be detected by binding with the other). “Addressable sets of probes” and analogous terms refer to the multiple regions of different moieties supported by or intended to be supported by the array surface.

The term “sample” as used herein relates to a material or mixture of materials, containing one or more components of interest. Samples include, but are not limited to, samples obtained from an organism or from the environment (e.g., a soil sample, water sample, etc.) and can be directly obtained from a source (e.g., such as a biopsy or from a tumor) or indirectly obtained e.g., after culturing and/or one or more processing steps. In some embodiments, samples are a complex mixture of molecules, e.g., comprising at least about 50 different molecules, at least about 100 different molecules, at least about 200 different molecules, at least about 500 different molecules, at least about 1000 different molecules, at least about 5000 different molecules, at least about 10,000 molecules, etc.

As used herein, a “biologically occurring sequence” refers to a sequence in a biological sample of target nucleic acids, e.g., such as a sequence from a biological organism, cell, tissue type, etc., being evaluated by hybridization to a collection of probe molecules which are designed to detect one or more sequences in the biological sample (e.g., by specifically hybridizing to the sequence under stringent conditions). Probes with no significant similarity to a biologically occurring sequence are those which are selected (e.g., by methods as described herein) not to hybridize to the sequences under stringent conditions such that they can be used as negative controls for test probes which are designed to detect the one or more sequences in the biological sample.

The term “genome” refers to all nucleic acid sequences (coding and non-coding) and elements present in any virus, single cell (prokaryote and eukaryote) or each cell type in a metazoan organism. The term genome also applies to any naturally occurring or induced variation of these sequences that can be present in a mutant or disease variant of any virus or cell or cell type. Genomic sequences include, but are not limited to, those involved in the maintenance, replication, segregation, and generation of higher order structures (e.g. folding and compaction of DNA in chromatin and chromosomes), or other functions, if any, of nucleic acids, as well as all the coding regions and their corresponding regulatory elements needed to produce and maintain each virus, cell or cell type in a given organism.

For example, the human genome consists of approximately 3.0×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell can contain variable numbers of each chromosome in addition to deletions, rearrangements and amplification of any subchromosomal region or DNA sequence. In some embodiments, a “genome” refers to nuclear nucleic acids, excluding mitochondrial nucleic acids; however, in some embodiments, the term does not exclude mitochondrial nucleic acids. In some embodiments, the “mitochondrial genome” is used to refer specifically to nucleic acids found in mitochondrial fractions.

By “genomic source” is meant the initial nucleic acids that are used as the original nucleic acid source from which the probe nucleic acids are produced, e.g., as a template in the nucleic acid amplification and/or labeling protocols.

As used herein, a “test nucleic acid sample” or “test nucleic acids” refer to nucleic acids comprising sequences whose quantity or degree of representation (e.g., copy number) or sequence identity is being assayed. Similarly, “test genomic acids” or a “test genomic sample” refers to genomic nucleic acids comprising sequences whose quantity or degree of representation (e.g., copy number) or sequence identity is being assayed.

If a surface-bound polynucleotide or probe “corresponds to” a chromosomal region, the polynucleotide usually contains a sequence of nucleic acids that is unique to that chromosomal region. Accordingly, a surface-bound polynucleotide that corresponds to a particular chromosomal region usually specifically hybridizes to a labeled nucleic acid made from that chromosomal region, relative to labeled nucleic acids made from other chromosomal regions.

As used herein, a “reference nucleic acid sample” or “reference nucleic acids” refers to nucleic acids comprising sequences whose quantity or degree of representation (e.g., copy number) or sequence identity is known. Similarly, “reference genomic acids” or a “reference genomic sample” refers to genomic nucleic acids comprising sequences whose quantity or degree of representation (e.g., copy number) or sequence identity is known. A “reference nucleic acid sample” can be derived independently from a “test nucleic acid sample,” i.e., the samples can be obtained from different organisms or different cell populations of the sample organism. However, in some embodiments, a reference nucleic acid is present in a “test nucleic acid sample” which comprises one or more sequences whose quantity or identity or degree of representation in the sample is unknown while containing one or more sequences (the reference sequences) whose quantity or identity or degree of representation in the sample is known. The reference nucleic acid can be naturally present in a sample (e.g., present in the cell from which the sample was obtained) or can be added to or spiked in the sample.

A “non-cellular chromosome composition” is a composition of chromosomes synthesized by mixing pre-determined amounts of individual chromosomes. These synthetic compositions can include selected concentrations and ratios of chromosomes that do not naturally occur in a cell, including any cell grown in tissue culture. Non-cellular chromosome compositions can contain more than an entire complement of chromosomes from a cell, and, as such, can include extra copies of one or more chromosomes from that cell. Non-cellular chromosome compositions can also contain less than the entire complement of chromosomes from a cell.

A “CGH array” or “aCGH array” refers to an array that can be used to compare DNA samples for relative differences in copy number. In general, an aCGH array can be used in any assay in which it is desirable to scan a genome with a sample of nucleic acids. For example, an aCGH array can be used in location analysis as described in U.S. Pat. No. 6,410,243, the entirety of which is incorporated herein and thus can also be referred to as a “location analysis array” or an “array for ChIP-chip analysis.” In some embodiments, a CGH array provides probes for screening or scanning a genome of an organism and comprises probes from a plurality of regions of the genome. In some embodiments, an array comprises probe sequences for scanning an entire chromosome arm, wherein probes targets are separated by at least about 500 bp, at least about 1 kb, at least about 5 kb, at least about 10 kb, at least about 25 kb, at least about 50 kb, at least about 100 kb, at least about 250 kb, at least about 500 kb and at least about 1 Mb. In some embodiments, an array comprises probe sequences for scanning an entire chromosome, a set of chromosomes, or the complete complement of chromosomes forming the organism's genome. By “resolution” is meant the spacing on the genome between sequences found in the probes on the array. In some embodiments (e.g., using a large number of probes of high complexity) all sequences in the genome can be present in the array. The spacing between different locations of the genome that are represented in the probes can also vary, and can be uniform, such that the spacing is substantially the same between sampled regions, or non-uniform, as desired. An assay performed at low resolution on one array, e.g., comprising probe targets separated by larger distances, can be repeated at higher resolution on another array, e.g., comprising probe targets separated by smaller distances.

In some embodiments, chromatin immunoprecipitation-chip (ChIP-chip) analysis involves methods of identifying a region of a genome of a living cell to which a protein of interest binds. The methods can comprise the steps of a) formaldehyde crosslinking DNA binding protein in the living cell to genomic DNA of the living cell, thereby producing DNA binding protein crosslinked to genomic DNA; b) generating DNA fragments of the genomic DNA crosslinked to DNA binding protein in a), thereby producing DNA fragments to which DNA binding protein is bound; c) immunoprecipitating the DNA fragment produced in b) to which the protein of interest is bound using an antibody that specifically binds the protein of interest; d) separating the DNA fragment identified in c) from the protein of interest; e) amplifying the DNA fragment of d) using ligation-mediated polymerase chain reaction; f) fluorescently labeling the DNA fragment of e); g) combining the labeled DNA fragment of e) with a DNA microarray comprising a sequence complementary to genomic DNA of the cell, under conditions in which hybridization between the DNA fragment and a region of the sequence complementary to genomic DNA occurs; h) identifying the region of the sequence complementary to genomic DNA to which the DNA fragment hybridizes by measuring the fluorescence intensity; and i) comparing the fluorescence intensity measured in h) to the fluorescence intensity of a control, whereby fluorescence intensity in a region of the genome which is greater than the fluorescence intensity of the control in the region indicates the region of the genome in the cell to which the protein of interest binds. In some embodiments, a nucleic acid construct as described herein is added prior to a processing step, such as prior to step (a), or prior to step (b).

In some embodiments, in constructing an array, both coding and non-coding genomic regions are included as probes, whereby “coding region” refers to a region comprising one or more exons that is transcribed into an mRNA product and from there translated into a protein product, while by non-coding region is meant any sequences outside of the exon regions, where such regions can include regulatory sequences, e.g., promoters, enhancers, untranslated but transcribed regions, introns, origins of replication, telomeres, etc. In some embodiments, one can have at least some of the probes directed to non-coding regions and others directed to coding regions. In some embodiments, one can have all of the probes directed to non-coding sequences and such sequences can, optionally, be all non-transcribed sequences (e.g., intergenic regions including regulatory sequences such as promoters and/or enhancers lying outside of transcribed regions).

In some embodiments, an array can be optimized for one type of genome scanning application compared to another, for example, an array can be enriched for intergenic regions compared to coding regions for a location analysis application.

In some embodiments, at least 5% of the polynucleotide probes on the solid support hybridize to regulatory regions of a nucleotide sample of interest while other embodiments can have at least 30% of the polynucleotide probes on the solid support hybridize to exonic regions of a nucleotide sample of interest. In some embodiments, at least 50% of the polynucleotide probes on the solid support hybridize to intergenic regions (e.g., non-coding regions which exclude introns and untranslated regions, i.e., comprise non-transcribed sequences) of a nucleotide sample of interest.

In some embodiments, probes on an array represent random selection of genomic sequences (e.g., both coding and noncoding). However, in some embodiments, particular regions of the genome are selected for representation on an array, e.g., such as genes belonging to particular pathways of interest or whose expression and/or copy number are associated with particular physiological responses of interest (e.g., disease, such a cancer, drug resistance, toxological responses and the like). In some embodiments, where particular genes are identified as being of interest, intergenic regions proximal to those genes are included on an array along with, optionally, all or portions of the coding sequence corresponding to the genes. In some embodiments, at least about 100 bp, 500 bp, 1,000 bp, 5,000 bp, 10,000 bp or even 100,000 bp of genomic DNA upstream of a transcriptional start site is represented on an array in discrete or overlapping sequence probes. In some embodiments, at least one probe sequence comprises a motif sequence to which a protein of interest (e.g., such as a transcription factor) is known or suspected to bind.

In some embodiments, repetitive sequences are excluded as probes on an array. However, in some embodiments, repetitive sequences are included.

The choice of nucleic acids to use as probes can be influenced by prior knowledge of the association of a particular chromosome or chromosomal region with certain disease conditions. International Application WO 93/18186 provides a list of exemplary chromosomal abnormalities and associated diseases, which are described in the scientific literature. Alternatively, whole genome screening to identify new regions subject to frequent changes in copy number can be performed using the methods presently disclosed.

In some embodiments, previously identified regions from a particular chromosomal region of interest are used as probes. In some embodiments, an array can include probes which “tile” a particular region (e.g., which have been identified in a previous assay or from a genetic analysis of linkage), by which is meant that the probes correspond to a region of interest as well as genomic sequences found at defined intervals on either side, i.e., 5′ and 3′ of, the region of interest, where the intervals may or may not be uniform, and can be tailored with respect to the particular region of interest and the assay objective. In other words, the tiling density can be tailored based on the particular region of interest and the assay objective. Such “tiled” arrays and assays employing the same are useful in a number of applications, including applications where one identifies a region of interest at a first resolution, and then uses tiled array tailored to the initially identified region to further assay the region at a higher resolution, e.g., in an iterative protocol.

In some embodiments, an array includes probes to sequences associated with diseases associated with chromosomal imbalances for prenatal testing. For example, in some embodiments, an array comprises probes complementary to all or a portion of chromosome 21 (e.g., Down's syndrome), all or a portion of the X chromosome (e.g., to detect an X chromosome deficiency as in Turner's Syndrome) and/or all or a portion of the Y chromosome Klinefelter Syndrome (to detect duplication of an X chromosome and the presence of a Y chromosome), all or a portion of chromosome 7 (e.g., to detect William's Syndrome), all or a portion of chromosome 8 (e.g., to detect Langer-Giedon Syndrome), all or a portion of chromosome 15 (e.g., to detect Prader-Willi or Angelman's Syndrome), all or a portion of chromosome 22 (e.g., to detect Di George's syndrome).

Other “themed” arrays can be fabricated, for example, arrays including probes for duplications or deletions associated with specific types of cancer (e.g., breast cancer, prostate cancer and the like). The selection of such arrays can be based on patient information such as familial inheritance of particular genetic abnormalities. In some embodiments, an array for scanning an entire genome is first contacted with a sample and then a higher-resolution array is selected based on the results of such scanning.

Themed arrays also can be fabricated for use in gene expression assays, for example, to detect expression of genes involved in selected pathways of interest, or genes associated with particular diseases of interest.

In some embodiments, a plurality of probes on an array are selected to have a duplex Tₘ within a predetermined range. For example, in some embodiments, at least about 50% of the probes have a duplex Tₘ within a temperature range of about 75° C. to about 85° C. In some embodiments, at least 80% of said polynucleotide probes have a duplex Tₘ within a temperature range of about 75° C. to about 85° C., within a range of about 77° C. to about 83° C., within a range of from about 78° C. to about 82° C. or within a range from about 79° C. to about 82° C. In some embodiments, at least about 50% of probes on an array have range of Tₘ's of less than about 4° C., less then about 3° C., or even less than about 2° C., e.g., less than about 1.5° C., less than about 1.0° C. or about 0.5° C.

The probes on the microarray, in some embodiments, have a nucleotide length in the range of at least 30 nucleotides to 200 nucleotides, or in the range of at least about 30 to about 150 nucleotides. In some embodiments, at least about 50% of the polynucleotide probes on the solid support have the same nucleotide length, and that length can be about 60 nucleotides.

In some embodiments, probes on an array comprise at least coding sequences.

In some embodiments, probes represent sequences from an organism such as Drosophila melanogaster, Caenorhabditis elegans, yeast, zebrafish, a mouse, a rat, a domestic animal, a companion animal, a primate, a human, etc. In some embodiments, probes representing sequences from different organisms are provided on a single substrate, e.g., on a plurality of different arrays.

Methods to fabricate arrays are described in detail in U.S. Pat. Nos. 6,242,266; 6,232,072; 6,180,351; 6,171,797 and 6,323,043. As already mentioned, these references are incorporated herein by reference. Drop deposition methods can be used for fabrication, as previously described herein. Also, instead of drop deposition methods, photolithographic array fabrication methods can be used. Interfeature areas need not be present particularly when an array is made by photolithographic methods as described in those patents.

Following receipt by a user, an array can be exposed to a sample and then read. Reading of an array can be accomplished by illuminating the array and reading the location and intensity of resulting fluorescence at multiple regions on each feature of the array. For example, a scanner can be used for this purpose, such as the AGILENT MICROARRAY SCANNER manufactured by Agilent Technologies (Santa Clara, Calif.) or other similar scanner. Other suitable apparatus and methods are described in U.S. Pat. Nos. 6,518,556; 6,486,457; 6,406,849; 6,371,370; 6,355,921; 6,320,196; 6,251,685 and 6,222,664. Scanning typically produces a scanned image of the array which can be directly inputted to a feature extraction system for direct processing and/or saved in a computer storage device for subsequent processing. However, arrays can be read by any other methods or apparatus than the foregoing, other reading methods including other optical techniques or electrical techniques (where each feature is provided with an electrode to detect bonding at that feature in a manner disclosed in U.S. Pat. Nos. 6,251,685, 6,221,583 and elsewhere).

It should also be noted that, as used in this specification and the appended claims, the term “configured” describes a system, apparatus, or other structure that is constructed or configured to perform a particular task or adopt a particular configuration to. The phrase “configured” can be used interchangeably with other similar phrases such as arranged and configured, constructed and arranged, adapted, constructed, manufactured and arranged, and the like.

The practice of the present methods can employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Some embodiments of suitable techniques can be had by reference to the examples hereinbelow. However, other equivalent conventional procedures can, of course, also be used. Such conventional techniques and descriptions can be found in standard laboratory manuals such as Genome Analysis: A Laboratory Manual Series (Vols. I-IV); Using Antibodies: A Laboratory Manual; Cells: A Laboratory Manual; PCR Primer: A Laboratory Manual; and Molecular Cloning: A Laboratory Manual (all from Cold Spring Harbor Laboratory Press); Stryer, “Biochemistry” (WH Freeman); Gait, “Oligonucleotide Synthesis: A Practical Approach” 1984, IRL Press, London; Freifelder, D “Molecular Biology” 2^{nd} edition, Jones & Bartlett (1987); Ausubel et al. eds., “Current Protocols in Molecular Biology”, chapters 1-3, John Wiley (1994) all of which are herein incorporated in their entirety by reference for all purposes.

Control Nucleic Acid Constructs

A control nucleic acid construct as described herein can be used as a reference to spike samples of nucleic acids, such as a test sample or a reference sample, prior to processing and analysis steps. In some embodiments, a control nucleic acid construct is used as an exogenous spike for a sample. For example, a control nucleic acid construct can be used as a normalization control in CGH experiments. A control nucleic acid construct can be used to assess system specificity, sensitivity, and dynamic range. A control nucleic acid construct can be used in assay development, in product development and validation, and for quality control. A “spike” or “spiking reagent” refers to a reagent having a known composition which can be added to a sample at a known concentration and which acts as an internal control during preparation and analysis to monitor method performance.

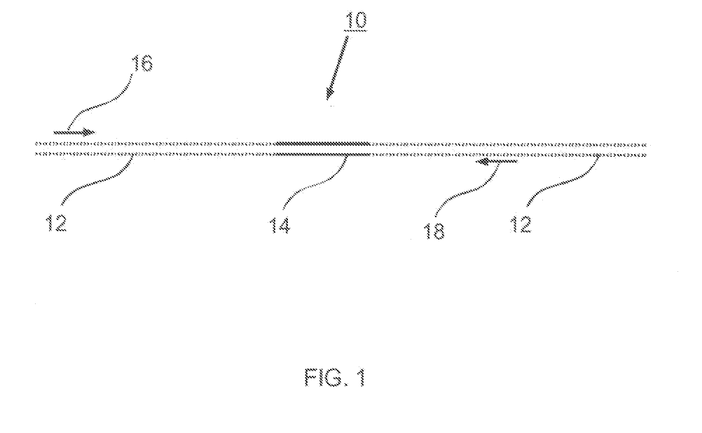

In some embodiments, a control nucleic construct 10 comprises a vector 12 having a control nucleic acid molecule 14 inserted therein (FIG. 1). In some embodiments, only a single control nucleic acid molecule is inserted. The sequence of the control nucleic acid as disclosed herein is complementary to a negative control probe, as described hereinbelow and in U.S. patent application Ser. No. 11/292,588, the disclosure of which is incorporated by reference herein. In some embodiments, a negative control probe is variable in sequence and matched in base content and melting temperature to other probes on a microarray but is not substantially complementary to nucleic acids expected to be in a sample under investigation, i.e., the probe does not hybridize to nucleic acids expected to be in a sample under investigation under stringent conditions.

The length of insert 14 can be selected as needed and will depend upon the length of the complementary negative control probe under consideration. In some embodiments, the length of a control nucleic acid can be in the range of 20 to 100 nucleotides, 10 to 200 nucleotides, or 10 to 500 nucleotides, for example. In some embodiments, the length of a control nucleic acid is 60 nucleotides. In some embodiments, the length of a control nucleic acid is 200 nucleotides. Non-limiting examples of control nucleic acids include SEQ ID NOs: 1-44 as shown in Table 1.

TABLE 1

| | | |
|---|---|---|
| SEQ ID | Orien- | |
| NO: | tation | Control Nucleic Acid Sequence |

| | | |
|---|---|---|
| 1 | 5′-3′ | GACTTAAATTCTTCATAACTCGACTACGAGACCTAATGTCGGACTAAGTTAACCAATAAA |
| 2 | 3′-5′ | CTGAATTTAAGAAGTATTGAGCTGATGCTCTGGATTACAGCCTGATTCAATTGGTTATTT |
| 3 | 5′-3′ | TTTGTAATCTCGATACGCGTAAGTTTCGATCAGGTAATTTACATCGACATAGACACCCTA |
| 4 | 3′-5′ | AAACATTAGAGCTATGCGCATTCAAAGCTAGTCCATTAAATGTAGCTGTATCTGTGGGAT |
| 5 | 5′-3′ | CGATAAAAAGTCATTGTATCGAGTGATACCGTAACCTACCGTTCGTAGACTATTATAAGA |
| 6 | 3′-5′ | GCTATTTTTCAGTAACATAGCTCACTATGGCATTGGATGGCAAGCATCTGATAATATTCT |
| 7 | 5′-3′ | TCTCGGTAAATAGAGTTTCGTGCTTATACTAGATGTAGTCTACGAGATAGACGCTAGATT |
| 8 | 3′-5′ | AGAGCCATTTATCTCAAAGCACGAATATGATCTACATCAGATGCTCTATCTGCGATCTAA |
| 9 | 5′-3′ | AAGTAACGTGAGTAGTATGATCATGTTACGCGAGGATCGTTATCGAGTTACAATAACATA |
| 10 | 3′-5′ | TTCATTGCACTCATCATACTAGTACAATGCGCTCCTAGCAATAGCTCAATGTTATTGTAT |
| 11 | 5′-3′ | TCGGGTTTACTTGATATCAAGCGCGGTTAGAATTGAATACGATGAGACGAATTTATTAGA |
| 12 | 3′-5′ | AGCCCAAATGAACTATAGTTCGCGCCAATCTTAACTTTGCTACTCTGCCTTAAATAATCT |
| 13 | 5′-3′ | ATACGAATCTTACGTAGTTTAGTGTCGCTTCACTAAAAGGCTCTATATTCGGATAGTGCA |
| 14 | 3′-5′ | TATGCTTAGAATGCATCAAATCACAGCGAAGTGATTTTCCGAGATATAAGCCTATCACGT |
| 15 | 5′-3′ | GGCTATCATAGAAATGTAGTCGAATCGTAGCATACTCGAATTAGATATCTCTATGCTAAG |
| 16 | 3′-5′ | CCGATAGTATCTTTACATCAGCTTAGCATCGTATGAGCTTAATCTATAGAGATACGATTC |
| 17 | 5′-3′ | CAACGTTGTTATACGTCGTTACCTCAAAATGCGCGTAAAAACCTGTGAACTATTATAAAG |
| 18 | 3′-5′ | GTTGCAACAATATGCAGCAATGGAGTTTTACGCGCATTTTTGGACACTTGATAATATTTC |
| 19 | 5′-3′ | TTGAACTTATGTAATCTGGTAGTATCGAGACAATCGTTACAGCGCCATATGTAATGAGAA |
| 20 | 3′-5′ | AACTTGAATACATTAGACCATCATAGCTCTGTTAGCAATGTCGCGGTATACATTACTCTT |
| 21 | 5′-3′ | TCGTGCAGACTTCTACAACATCGAGTTCTGCAACGTAATAACCGTATGAATAAGACTAGT |
| 22 | 3′-5′ | AGCACGTCTGAAGATGTTGTAGCTCAAGACGTTGCATTATTGCCATACTTATTCTGATCA |
| 23 | 5′-3′ | CTGGTCTTAATCGTCTTGTTAACTAATACGGGCATTTACGAGTCGATAGACATATAATCA |
| 24 | 3′-5′ | GACCAGAATTAGCAGAACAATTGATTATGCCCGTAAATGCTCAGCTATCTGTATATTAGT |
| 25 | 5′-3′ | TGACAACTAGTTTGCAATCGTTATAAGTCGTATTAACGCGAAATTAACCTGCTAGGAACT |
| 26 | 3′-5′ | ACTGTTGATCAAACGTTAGCAATATTCAGCATAATTGCGCTTTAATTGGACGATCCTTGA |
| 27 | 5′-3′ | ATTAGAACTACTATAAATCCGGCGAGATTCTATGGCGCATAACATGATAGACAGAACATT |
| 28 | 3′-5′ | TAATCTTGATGATATTTAGGCCGCTCTAAGATACCGCGTATTGTACTATCTGTCTTGTAA |
| 29 | 5′-3′ | GTTACCGTTTGAATAATAACGGACGGATAACCCTTTGATACATCCCAACGTATAATAAGG |
| 30 | 3′-5′ | CAATGGCAAACTTATTATTGCCTGCCTATTGGGAAACTATGTAGGGTTGCATATTATTCC |
| 31 | 5′-3′ | GTAGAGTATATTGCTTTAATACGACCCCGATAAGCACGATCGTATTAGACATAGATGATA |
| 32 | 3′-5′ | CATCTCATATAACGAAATTATGCTGGGGCTATTCGTGCTAGCATAATCTGTATCTACTAT |
| 33 | 5′-3′ | ATAATTCGTTGACTATAGCACATTTCGATCCTCGTTATGATACCAATGAACGGAAGTCTT |
| 34 | 3′-5′ | TATTAAGCAACTGATATCGTGTAAAGCTAGGAGCAATACTATGGTTACTTGCCTTCAGAA |
| 35 | 5′-3′ | CAGATCGATCGGTTTATATGCGATTTAACGCCGCTTTCATCCTAAAGCGCAAATTTTACA |
| 36 | 3′-5′ | GTCTAGCTAGCCAAATATACGCTAAATTGCGGCGAAAGTAGGATTTCGCGTTTAAAATGT |
| 37 | 5′-3′ | TACGTCAATTCGTGATATGCCTTTCGATTATCATACCGAAGAGTCCTTTAGTAAGTTTAG |
| 38 | 3′-5′ | ATGCAGTTAAGCACTATACGGAAAGCTAATAGTATGGCTTCTCAGGAAATCATTCAAATC |
| 39 | 5′-3′ | GAAACTAGTGAAACAGAGTTCGCTAAGCGTCTAAACTCGAGTTTTTACGAACTAATACAA |
| 40 | 3′-5′ | CTTTGATCACTTTGTCTCAAGCGATTCGCAGATTTGAGCTCAAAAATGCTTGATTATGTT |
| 41 | 5′-3′ | GGTATTGTTCTTATATTCATCGTGACCAGTAACCAATTGATATCGGATTTCGGTTTACAG |
| 42 | 3′-5′ | CCATAACAAGAATATAAGTAGCACTGGTCATTGGTTAACTATAGCCTAAAGCCAAATGTC |
| 43 | 5′-3′ | CTATTTCTCGAAACCGTTAAATCGAAATGTTATGTCCGCTAATCGAACCACTAATCGTTT |
| 44 | 3′-5′ | GATAAAGAGCTTTGGCAATTTAGCTTTACAATACAGGCGATTAGCTTGGTGATTAGCAAA |

In Table 1, a plus strand is listed above its reverse-complement strand (minus strand). A control nucleic acid molecule as described herein can comprise a duplex of such plus and minus strands. A negative control probe, as described herein, can comprise a sequence that is complementary to either of these strands. As a non-limiting example, a control nucleic acid molecule can comprise a nucleic acid having the sequence identified by SEQ ID NO:1, and the corresponding negative control probe would correspond to the sequence identified by SEQ ID NO:2.

In some embodiments, the length of a control nucleic acid construct can be in the range of 2 to 10 kilobases, 10 to 20 kilobases, 10 to 50 kilobases, or 10 to 100 kilobases, for example. In some embodiments, the length of a control nucleic acid construct can be greater than 2 kilobases, greater than 10 kilobases, greater than 50 kilobases, greater than 100 kilobases, or longer. In some embodiments, a control nucleic acid construct as described herein does not include at least one of the following: a homopolymeric run, a poly-A sequence, a T3 promoter site, a T7 promoter, a Tag sequence, a concatenated sequence, concatenated Tag sequences, and an RNA promoter (see, e.g., U.S. Patent Application Publication No. 2004/0175719).

A control nucleic acid can be prepared using any suitable method, such as, for example, the known phosphotriester and phosphite triester methods, or automated embodiments thereof. In one such automated embodiment, dialkyl phosphoramidites are used as starting materials and can be synthesized as described by Beaucage et al. (1981) Tetrahedron Letters 22:1859. A non-limiting exemplary method for synthesizing oligonucleotides on a modified solid support is described in U.S. Pat. No. 4,458,066. Chemical synthesis of DNA can be accomplished using a commercial DNA synthesizer such as for example a DNA synthesizer using the thiophosphate method (Shimazu) or a DNA synthesizer using the phosphoamidite method (Perkin Elmer).

A control nucleic acid construct can be prepared by incorporating a double-stranded control nucleic acid molecule into an appropriate cloning vector. E. coli or other host cells are transformed using the recombinant vector, and positive transformants are selected using tetracycline resistance or ampicillin resistance as the marker. The cloning vector for preparing the control nucleic acid construct may be any vector capable of independent replication in host cells, and for example a phage vector, plasmid vector or the like can be used. Escherichia coli cells or the like for example can be used as the host cells.

Transformation of E. coli or other host cells can be accomplished for example by a method of adding the recombinant vector to competent cells prepared in the presence of calcium chloride, magnesium chloride or rubidium chloride. When a plasmid is used as the vector, it is desirable to include therein a tetracycline, ampicillin or other drug-resistance gene.

In some embodiments, to prepare a recombinant vector, a nucleic acid fragment (e.g., DNA fragment) of a suitable length is prepared which comprises the control nucleic acid. A recombinant vector is prepared by inserting this nucleic acid fragment downstream from the promoter of an appropriate expression vector, and this recombinant vector is introduced into appropriate host cells. The aforementioned nucleic acid fragment is incorporated into the vector so that it may be cloned. In addition to the promoter the vector may contain enhancers and other cis-elements, splicing signals, poly A addition signals, selection markers (such as the dihydrofolic acid reductase gene, ampicillin resistance gene or neomycin resistance gene), ribosome binding sequences (SD sequences) and the like.

Any suitable expression vector can be used in making a control nucleic acid construct as described herein as long as the vector does not have a sequence that interferes with processing or analysis steps as described herein. A vector is generally considered to be an agent that can carry a DNA fragment into a host cell. A wide variety of vectors are available. There are no particular limits on the expression vector as long as it is capable of independent replication in the host cells, and for example plasmid vectors, phage vectors, virus vectors and the like can be used. Non-limiting examples of vectors include single-stranded, double-stranded, linear, or circular molecules. The vector can be a viral nucleic acid. Non-limiting embodiments of suitable vectors include EIA adenovirus, filamentous phage, phage, cosmid, YAC, and lambda phage. Other examples include lambda gt11 (Stratagene; and see, e.g., Young et al. (1983) Proc. Nat. Acad. Sci. USA 80:1194-1198), lambda ZAP, lambda ZAP, lambda DASH, lambda gt101, pDrive Cloning Vector (Qiagen), N15, pQE-30 UA vector, Flexi, pCAT-3, pGEM, PGL2, PG51uc, PGL3, PSP, M13, and PBR322. Non-limiting examples of plasmid vectors include E. coli-derived plasmids (such as pRSET, pBR322, pBR325, pUC118, pUC119, pUC18 and pUC19), B. subtilis-derived plasmids (such as pUB110 and pTP5) and yeast-derived plasmids (such as YEp13, YEp24 and YCp50), examples of phage vectors include gamma-phages (such as Charon4A, Charon21A, EMBL3, EMBL4, gamma-gt10, gamma-gt11 and gamma-ZAP), and examples of virus vectors include animal viruses including retroviruses, vaccinia virus and the like and insect viruses such as baculoviruses and the like.

Any of prokaryotic cells, yeasts, animal cells, insect cells, plant cells or the like can be used as the host cells as long as they can express the control nucleic acid. Individual animals, plants, silkworms or the like can also be used.

When using bacterial cells as host cells, for example Escherichia coli or other Escherichia, Bacillus subtilis or other Bacillus, Pseudomonas putida or other Pseudomonas or Rhizobium meliloti or other Rhizobium bacteria can be used as the host cells. Specifically, E. coli such as Escherichia coli XL1-Blue, Escherichia coli XL2-blue, Escherichia coli DH1, Escherichia coli K12, Escherichia coli JM109, Escherichia coli HB101 or the like or Bacillus subtilis such as Bacillus subtilis MI114, Bacillus subtilis 207-21 or the like can be used. There are no particular limits on the promoter in this case as long as it is capable of expression in E. coli or other bacteria, and for example a trp promoter, lac promoter, PL promoter, PR promoter or other E. coli- or phage-derived promoter can be used. An artificially designed and modified promoter such as a tac promoter, lac T7 promoter or let I promoter can also be used.

There are no particular limits on the method of introducing the recombinant vector into the bacteria as long as it is a method capable of introducing DNA into bacteria, and for example electroporation or a method using calcium ions or the like can be used.

When using yeasts as host cells, for example Saccharomyces cerevisiae, Schizosaccharomyces pombe, Pichia pastoris or the like can be used as the host cells. There are no particular limits on the promoter in this case as long as it can be expressed in yeasts, and for example a gall promoter, gal10 promoter, heat shock protein promoter, MFα1 promoter, PHO5 promoter, PGK promoter, GAP promoter, ADH promoter, AOX1 promoter or the like can be used.

There are no particular limits on the method of introducing the recombinant vector into the yeast as long as it is a method capable of introducing DNA into yeast, and for example the electroporation method, spheroplast method, lithium acetate method or the like can be used.

When using animal cells as host cells, for example monkey COS-7 cells, Vero cells, chinese hamster ovary cells (CHO cells), mouse L cells, rat GH3, human FL cells or the like can be used as the host cells. There are no particular limits on the promoter in the case as long as it can be expressed in animal cells, and for example an SR.alpha. promoter, SV40 promoter, LTR (long terminal repeat) promoter, CMV promoter, human cytomegalovirus initial gene promoter or the like can be used.

There are no particular limits on the method of introducing the recombinant vector into the animal cells as long as it is a method capable of introducing DNA into animal cells, and for example the electroporation method, calcium phosphate method, lipofection method or the like can be used.

When using insect cells as host cells, for example Spodoptera frugiperda ovary cells, Trichoplusia in ovary cells, cultured cells derived from silkworm ovaries or the like can be used as the host cells. Examples of Spodoptera frugiperda ovary cells include Sf9, Sf21 and the like, examples of Trichoplusia ni ovary cells include High 5, BTI-TN-5B1-4 (Invitrogen) and the like, and examples of cultured cells derived from silkworm ovaries include Bombyx mori N4 and the like.

There are no particular limits on the method of introducing the recombinant vector into the insect cells as long as it is a method capable of introducing DNA into insect cells, and for example the calcium phosphate method, lipofection method, electroporation method or the like can be used.

A transformant into which has been introduced a recombinant vector having incorporated control nucleic acid is cultured by conventional culture methods. Culture of the transformant can be accomplished according to normal methods used in culturing host cells.

For the medium for culturing a transformant obtained as E. coli, yeast or other microbial host cells, either a natural or synthetic medium can be used as long as it contains carbon sources, nitrogen sources, inorganic salts and the like which are convertible by the microorganism and is a medium suitable for efficient culture of the transformant.

Glucose, fructose, sucrose, starch and other carbohydrates, acetic acid, propionic acid and other organic acids, and ethanol, propanol and other alcohols can be used as carbon sources. Ammonia, ammonium chloride, ammonium sulfate, ammonium acetate, ammonium phosphate and other ammonium salts of inorganic or organic acids and peptone, meat extract, yeast extract, corn steep liquor, casein hydrolysate and the like can be used as nitrogen sources. Monopotassium phosphate, dipotassium phosphate, magnesium phosphate, magnesium sulfate, sodium chloride, ferrous sulfate, manganese sulfate, copper sulfate, calcium carbonate and the like can be used as inorganic salts.

Culture of a transformant obtained as E. coli, yeast or other microbial host cells can be accomplished under aerobic conditions such as a shaking culture, aerated agitation culture or the like. The culture temperature is normally 25 to 37° C., the culture time is normally 12 to 48 hours, and the pH is maintained at 6 to 8 during the culture period. pH can be adjusted using inorganic acids, organic acids, alkaline solution, urea, calcium carbonate, ammonia or the like. Moreover, antibiotics such as ampicillin, tetracycline and the like can be added to the medium as necessary for purposes of culture.

When culturing a microorganism transformed with an expression vector using an inducible promoter as the promoter, an inducer can be added to the medium as necessary. For example, isopropyl-beta-D-thiogalactopyranoside or the like can be added to the medium when culturing a microorganism transformed with an expression vector using a lac promoter, and indoleacrylic acid when culturing a microorganism transformed with an expression vector using a trp promoter.

Commonly used RPMI1640 medium, Eagle's MEM medium, DMEM medium, Ham F12 medium, Ham F12K medium or a medium comprising one of these media with fetal calf serum or the like added can be used as the medium for culturing a transformant obtained with animal cells as the host cells. The transformant is normally cultured for 3 to 10 days at 37° C. in the presence of 5% CO₂. Moreover, an antibiotic such as kanamycin, penicillin, streptomycin or the like can be added as necessary to the medium for purposes of culture.

Transformants which can use commonly used TNM-FH medium (Pharmingen), Sf-900 II SFM medium (Gibco-BRL), ExCell400, ExCell405 (JRH Biosciences) or the like as the medium for culturing a transformant obtained with insect cells as the host cells are normally cultured for 3 to 10 days at 27° C. An antibiotic such as gentamicin or the like can be added to the medium as necessary for purposes of culture.

A control nucleic acid construct as described herein can be cloned and purified using conventional methods. Any suitable means can be used to insert a control nucleic acid into a vector. In some embodiments, a control nucleic acid strand plus and its reverse-complement strand are synthesized to include additional terminal bases which can be used, after the strands are annealed, to create an overhang which will facilitate ligation into a vector restriction site. For example, a sequence that will recreate a restriction endonuclease site can be incorporated into terminal sequences of control nucleic acid strands facilitating insertion into a vector that has been cleaved with the restriction endonuclease (such as, e.g., EcoR1). Preparation of DNA from bacteria can be accomplished using standard methods (see, e.g., Ausubel, et al.). Lipid and protein can be removed by digestion with proteinase K. Cell wall debris, polysaccharides, and remaining proteins can be removed by selective precipitation with cetyltrimethylammonium bromide (CTAB), and high molecular weight DNA can be recovered from the resulting supernatant by isopropanol precipitation. A cesium chloride gradient may also be utilized. Agarose gel electrophoresis can also be used in the purification.

In some embodiments, the complete sequence of a control nucleic acid construct is used in the methods described herein. In some embodiments a region (section) of a control nucleic acid construct is amplified to produce an amplicon (amplification product) comprising a control nucleic acid molecule, and the amplicon is used in the methods described herein. In some embodiments, the length of the amplicon can be in the range of about 0.5 kb (kilobases) to about 10 kb, about 1 to about 5 kb, or about 0.5 to about 2 kb. Any suitable amplification method can be used. An exemplary method is polyermase chain reaction (PCR). PCR is well known in the biotechnology art and is described in detail in U.S. Pat. No. 4,683,202; Eckert et al., The Fidelity of DNA polymerases Used In The Polymerase Chain Reactions, McPherson, Quirke, and Taylor (eds.), “PCR: A Practical Approach”, IRL Press, Oxford, Vol. 1, pp. 225-244; Andre, et. al. (1977) GENOME RESEARCH, Cold Spring Harbor Laboratory Press, pp. 843-852. In some embodiments, there are provided herein PCR primers capable of amplifying a region of a control nucleic acid construct wherein the region comprises a control nucleic acid molecule. A pair of such primers is shown schematically at 16 and 18 in FIG. 1. Non-limiting examples of forward, and reverse PCR primers capable of amplifying a sequence inserted into the EcoR1 site of Lambda gt11 include the following:

| | |
|---|---|
| CTGGATGTCGCTCCACAAA | SEQ ID NO: 45 |
| TTGATCGCCAGATAGTGGTGCTTC | SEQ ID NO: 46 |

In some embodiments, collections of different control nucleic acid constructs are provided. In some embodiments, the same vector is used, but with control nucleic acids having differing sequences inserted into each of the different constructs in the collection. In some embodiments, the length of each of the different control nucleic acids in the collection is the same. In some embodiments, the different control nucleic acid constructs are present in the collection at the same concentration. In some embodiments, the concentrations of at least two of the different control nucleic acid constructs in the collection differ. In some embodiments, the concentrations of at least some of the different control nucleic constructs in the collection span a range of concentrations. In some embodiments, the concentrations span 1, 2, 3, 4, 5, 6 or more orders of magnitude.

A control nucleic acid construct can be used as a spiking reagent in methods that utilize genomic DNA. In some embodiments, the complete sequence of the control nucleic acid construct is used as a spiking reagent. In some embodiments, a subsequence is used as a spiking reagent, and can be prepared, for example, by PCR amplification of a region of the complete control nucleic acid construct as described hereinabove. In some embodiments, the length of a spiking reagent is in the range of about 50% to 200% of the length of the nucleic acids in the sample being analyzed. In some embodiments, the length of a spiking reagent is in the range of about 10% to about 50%, about 10% to about 200%, about 50% to about 150%, or about 80% to about 120% of the length of the nucleic acids in the sample being analyzed. In some embodiments, the length of a spiking reagent is in the range of about 10% to about 200% of the length of the nucleic acids in the sample being analyzed. In some embodiments, the length of a spiking reagent is about 100% of the length of the nucleic acids in the sample being analyzed.

In some embodiments, a control nucleic acid construct as described herein can be used as a spiking reagent in methods that employ one or more sample preparation and analysis steps, such as, for example, protein-nucleic acid cross-linking, nucleic acid fragmentation, amplification, labeling, and microarray hybridization. Cross-linking can be achieved via chemical treatment, such as formaldehyde. Fragmentation can be accomplished by various means, such as, for example, sonication, shearing, or digestion with restriction endonucleases. Other processing steps can include amplification (such as, for example, PCR, LM-PCR, amplification using universal primers, amplification using processive DNA polymerases) and labeling steps, in which a nucleic acid is modified with a detectable label. For example, a control nucleic acid construct as described herein can be mixed with a nucleic acid sample, and the mixture subjected to one or more steps such as cross-linking, immunoprecipitation, fragmentation, amplification, labeling, and array hybridization.

Many genomic and genetic studies are directed to the identification of differences in gene dosage or expression among cell populations for the study and detection of disease. For example, many malignancies involve the gain or loss of DNA sequences resulting in activation of oncogenes or inactivation of tumor suppressor genes. Identification of the genetic events leading to, neoplastic transformation and subsequent progression can facilitate efforts to define the biological basis for disease, improve prognostication of therapeutic response, and permit earlier tumor detection. In addition, perinatal genetic problems frequently result from loss or gain of chromosome segments such as trisomy 21 or the micro deletion syndromes. Thus, methods of prenatal detection of such abnormalities can be helpful in early diagnosis of disease.

As mentioned hereinabove, comparative genomic hybridization (CGH) is one approach that has been employed to detect the presence and identify the location of amplified or deleted sequences. CGH reveals increases and decreases irrespective of genome rearrangement. In one implementation of CGH, genomic DNA is isolated from normal reference cells, as well as from test cells (e.g., tumor cells). The two nucleic acids are differentially labeled and then hybridized in situ to metaphase chromosomes of a reference cell. The repetitive sequences in both the reference and test DNAs are either removed or their hybridization capacity is reduced by some means. Chromosomal regions in the test cells which are at increased or decreased copy number can be identified by detecting regions where the ratio of signal from the two DNAs is altered. For example, those regions that have been decreased in copy number in the test cells will show relatively lower signal from the test DNA than the reference compared to other regions of the genome. Regions that have been increased in copy number in the test cells will show relatively higher signal from the test DNA.

In a variation of the above traditional CGH approach, the immobilized chromosome element can be replaced with a collection of solid support bound probe nucleic acids, e.g., an array of cDNAs.

In some embodiments, a control nucleic acid construct is used in a comparative genomic hybridization assay (CGH assay). In some embodiments, a population of nucleic acids contacted with an aCGH array comprises at least two sets of nucleic acid populations, which can be derived from different sample sources. For example, in some embodiments, a target population contacted with the array comprises a set of target molecules from a reference sample and from a test sample. In some embodiments, the reference sample is from an organism having a known genotype and/or phenotype, while the test sample has an unknown genotype and/or phenotype or a genotype and/or phenotype that is known and is different from that of the reference sample. For example, in some embodiments, the reference sample is from a healthy patient while the test sample is from a patient suspected of having cancer or known to have cancer.

In some embodiments, a target population being contacted to an array in a given assay comprises at least two sets of target populations that are identically labeled (i.e., are labeled with the same detectable label). In some embodiments, a target population being contacted to an array in a given assay comprises at least two sets of target populations that are differentially labeled (e.g., by spectrally distinguishable labels). In some embodiments, control target molecules in a target population are provided as two sets, e.g., a first set labeled with a first label and a second set labeled with a second label corresponding to first and second labels being used to label reference and test target molecules, respectively.

In some embodiments, the reference target molecules in a population are present at a level comparable to a haploid amount of a gene represented in the target population. In some embodiments, the reference target molecules are present at a level comparable to a diploid amount of a gene. In some embodiments, the reference target molecules are present at a level that is different from a haploid or diploid amount of a gene represented in the target population. The relative proportions of complexes formed labeled with the first label vs. the second label can be used to evaluate relative copy numbers of targets found in the two samples.

In some embodiments, the test and reference populations of nucleic acids can be mixed, such as in known proportions, and collection applied to an array. In some embodiments, test and reference populations of nucleic acids can be applied separately to separate but identical arrays (e.g., having identical probe molecules) and the signals from each array can be compared to determine relative copy numbers of the nucleic acids in the test and reference populations.

In practicing some embodiments of the subject methods, an initial step is to provide a genomic template. By genomic template is meant the nucleic acids that are used as template in primer extension reactions as described herein. In some embodiments, the genomic template is a population of genomic deoxyribonucleic acid molecules, where by population is meant a collection of molecules in which at least two constituent members have nucleotide sequences that differ from each other, e.g., by at least about 1 basepair, by at least about 5 basepairs, by at least about 10 basepairs, by at least about 50 base pairs, by at least about 100 base pairs, by at least about 1 kb, by at least about 10 kb etc. The number of distinct sequences in a population of molecules making up a given genomic template can be at least 2, at least 10 or at least 50, where the number of distinct molecules can be 1000, 5000, 10000, 100000 or higher.

The genomic template can be prepared using any convenient protocol. In many embodiments, the genomic template is prepared by first obtaining a source of genomic DNA, e.g., a nuclear fraction of a cell lysate, where any convenient means for obtaining such a fraction can be employed and numerous protocols for doing so are well known in the art. The genomic template can be genomic DNA representing the entire genome from a particular organism, tissue or cell type or can comprise a portion of the genome, such as a single chromosome. Genomic template can be prepared from a subject, for example a plant or an animal, that is suspected of being homozygous or heterozygous for a deletion or amplification of a genomic region. In many embodiments, the average size of the constituent molecules that make up the genomic template do not exceed about 10 kb in length, typically do not exceed about 8 kb in length and sometimes do not exceed about 5 kb in length, such that the average length of molecules in a given genomic template composition can range from about 1 kb to about 10 kb, usually from about 5 kb to about 8 kb in some embodiments. The genomic template can be prepared from an initial chromosomal source by fragmenting the source into the genomic template having molecules of the desired size range, where fragmentation can be achieved using any convenient protocol, including but not limited to: mechanical protocols, e.g., sonication, shearing, etc., chemical protocols, e.g., enzyme digestion, etc.

Following preparation of the genomic template, as described above, the prepared genomic template can be employed in the preparation of labeled probe nucleic acids using any suitable protocol (see, e.g., U.S. Pat. Nos. 7,011,949; 6,335,167; 6,197,501; 5,830,645; and 5,665,549; and U.S. Pat. Publication No. 20060094022). Protocols which utilize exo-Klenow fragment of DNA polymerase I and random primers are commercially available (e.g., BioPrimer Array CGH Labeling kit, (Invitrogen)).

Primer extension reactions for generating labeled nucleic acids are well known to those of skill in the art, and any convenient protocol can be employed. The primer is contacted with the template under conditions sufficient to extend the primer and produce a primer extension product. Primers are contacted with the genomic template in the presence of a sufficient DNA polymerase under primer extension conditions sufficient to produce the desired primer extension molecules. DNA polymerases of interest include, but are not limited to, polymerases derived from E. coli, thermophilic bacteria, archaebacteria, phage, yeasts, Neurosporas, Drosophilas, primates and rodents, likewise they include polymerases such as Reverse Transcriptases and the like. The DNA polymerase extends the primer according to the genomic template to which it is hybridized in the presence of additional reagents which include, but are not limited to: dNTPs; monovalent and divalent cations, e.g. KCl, MgCl₂; sulfhydryl reagents, e.g. dithiothreitol; and buffering agents; e.g. Tris-Cl.

Reagents employed in a primer extension reaction can include a labeling reagent, where the labeling reagent is often a labeled oligonucleotide, which can be labeled with a directly or indirectly detectable label. A directly detectable label is one that can be directly detected without the use of additional reagents, while an indirectly detectable label is one that is detectable by employing one or more additional reagent, e.g., where the label is a member of a signal producing system made up of two or more components. In many embodiments, the label is a directly detectable label, such as a fluorescent label, where the labeling reagent employed in such embodiments is a fluorescently tagged nucleotide(s), e.g. dCTP. Fluorescent moieties which can be used to tag nucleotides for producing labeled probe nucleic acids include, but are not limited to: fluorescein, the cyanine dyes, such as Cy3, Cy5, Alexa 555, Bodipy 630/650, and the like. Other labels can also be employed as are known in the art.

In some embodiments, a known amount of a control nucleic acid construct can be added to reference target molecules prior to processing and analysis steps. A known amount of the same control nucleic acid construct can be added to test target molecules prior to processing and analysis steps. In some embodiments, the amount of the control nucleic acid construct added to test target molecules and to reference molecules is the same. In some embodiments, the amount of the control nucleic acid construct added to test target molecules and to reference molecules is different.

In some embodiments, a first collection of different control nucleic acid constructs, with a known amount of each construct, can be added to reference target molecules prior to processing and analysis steps. A second collection of different control nucleic acid constructs, with a known amount of each construct, can be added to test target molecules prior to processing and analysis. In some embodiments, the control nucleic acid constructs in the first collection and in the second collection are the same. In some embodiments, the first collection and the second collection are identical. In some embodiments, the concentration of each of the different control nucleic acid constructs is the same in the first collection. In some embodiments, in the second collection the concentration of each of the different control nucleic acid constructs is the same. In some embodiments, the concentration of at least some of the different control nucleic acid constructs in the first collection is different. In some embodiments, the concentration of at least some of the different control nucleic acid constructs in the second collection is different. In some embodiments, the concentrations of the different control nucleic acid constructs in the first collection span a range of concentrations. In some embodiments, the concentrations of the different control nucleic acid constructs in the second collection span a range of concentrations.

In carrying out a hybridization analysis, an enormous number of array designs are possible. In some embodiments, a high density array will include a number of probes that specifically hybridize to the nucleic acids in a sample under analysis. In addition, the array can include one or more negative control probes as described hereinbelow. A control nucleic acid which is inserted into a control nucleic acid construct, as described herein, is perfectly complementary to a negative control probe.

The signal obtained from binding of labeled control nucleic acid can provide a control for variations in hybridization conditions, label intensity, reading efficiency, linearity of signal response, and other factors that can cause the signal of a perfect hybridization to vary between arrays. Gradient effects or “trends” are those in which there is a pattern of expression signal intensity which corresponds with specific physical locations on the substrate of the array and which may typically be characterized by a smooth change in the expression values from one location on the array to another. The signal obtained from binding of a labeled control nucleic acid can provide a control for monitoring the uniformity of a microarray, and can be used detrending signal intensity data. In some embodiments, signals read from all other probes in the array can be divided by the signal from the negative control probes thereby normalizing the measurements. Since the control nucleic acid construct is present during processing steps, it can aid in the evaluation of the overall process.

Negative control probes can be localized at any position in an array or at a multiple positions throughout the array to control for spatial variation in hybridization efficiency. In some embodiments, the negative control probes are located at the corners or edges of the array as well as in the middle. In some embodiments, an array can be divided into a plurality of quadrants or areas, and one or more negative control probes can be randomly located within each of the quadrants or areas.

Negative Control Probes

Some embodiments of methods disclosed herein can be used to generate negative control probe sequences. The term “negative control probe sequence” as used herein includes sequences of bases that can be deposited on an array and serve as a negative control during use of the array. The sequence is not limited by the type of application being performed, i.e., the sequence can be designed for arrays designed for any of a variety of uses, e.g., gene expression analysis, mutation analysis, sequencing, genotyping, comparative genome hybridization analysis, location analysis (e.g., ChIP-chip analysis), and genome scanning applications generally.

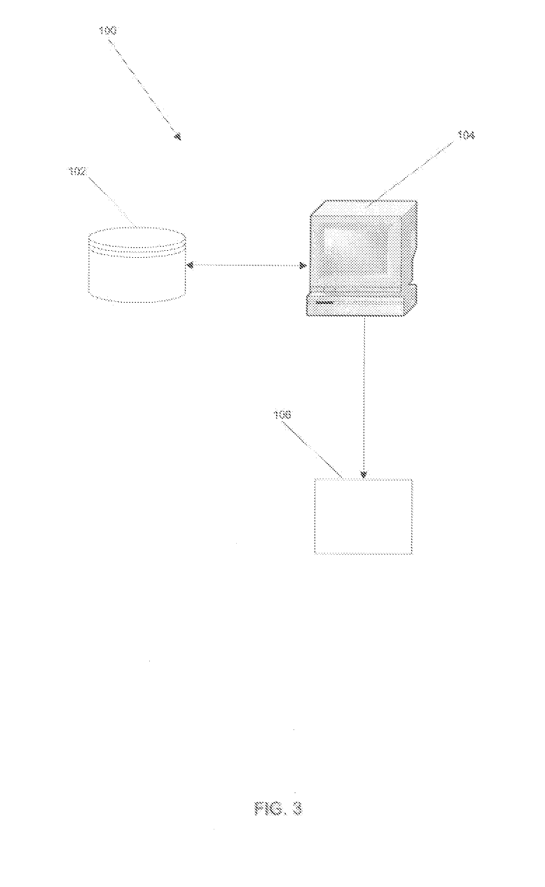

Referring now to FIG. 3, a schematic diagram of an exemplary system 100 for manufacturing arrays is shown. A computing system 104 is in electronic communication with a database 102 and an array printer 106. In some embodiments, the computing system 104 directs the operations of the array printer 106. It will be appreciated that in some embodiments the computing system 104 is part of the array printer 106. However, in some embodiments, the computing system 104 and the array printer 106 are separate. In addition, it will be appreciated that in some embodiments the database 102 is part of the computing system 104. However, in some embodiments, the database 102 and the computing system 104 are separate. The computing system 104 can query the database 102 as desired to retrieve data on probe sequences or on known sequences.

The array printer 106 can perform various steps to generate features of biopolymer probes (e.g., nucleic acids) on the array substrate. Exemplary array manufacturing machines and methods are described in U.S. Pat. Nos. 6,900,048; 6,890,760; 6,884,580; and 6,372,483. In some embodiments, the array printer 106 uses inkjet technology. In some embodiments, the array printer 106 prints spots of pre-synthesized nucleotide sequences onto the array substrate. In some embodiments, the array printer 106 can be used for in situ fabrication, where nucleotide sequences are built on the array one base at a time. Embodiments of the array printer 106 can also include those that use photolithographic methods to deposit nucleotide sequences onto the array substrates. Some embodiments of methods described herein are performed as a part of the array manufacturing process. However, some embodiments of methods described herein are performed separately from the array manufacturing process.

Some embodiments described herein are implemented as logical operations in a computing system, such as the computing system 104. The logical operations can be implemented (1) as a sequence of computer implemented steps or program modules running on a computer system and (2) as interconnected logic or hardware modules running within the computing system. This implementation is a matter of choice dependent on the performance requirements of the specific computing system. Accordingly, the logical operations making up the embodiments described herein are referred to as operations, steps, or modules. It will be recognized by one of ordinary skill in the art that these operations, steps, and modules can be implemented in software, in firmware, in special purpose digital logic, and any combination thereof without deviating from the spirit and scope of the claims attached hereto. This software, firmware, or similar sequence of computer instructions can be encoded and stored upon computer readable storage medium and can also be encoded within a carrier-wave signal for transmission between computing devices.

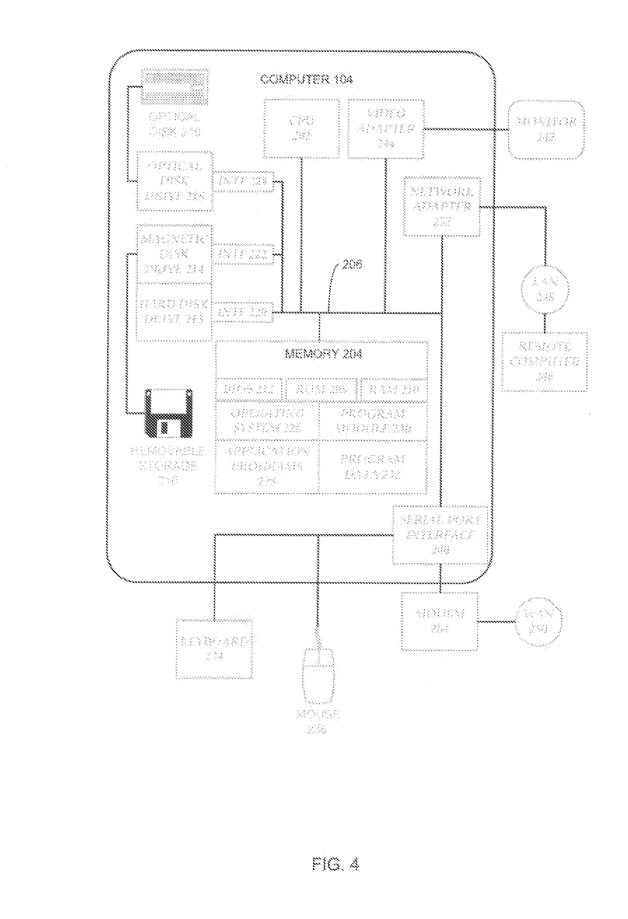

Referring now to FIG. 4, an example computing system 104 is illustrated. The computing system 104 illustrated in FIG. 4 can take a variety of forms such as, for example, a mainframe, a desktop computer, a laptop computer, a hand-held computer, or any other programmable device. In addition, although computing system 104 is illustrated, the systems and methods disclosed herein can be implemented in various alternative computer systems as well.

The computing system 104 includes a processor unit 202, a system memory 204, and a system bus 206 that couples various system components including the system memory 204 to the processor unit 202. The system bus 206 can be any of several types of bus structures including a memory bus, a peripheral bus and a local bus using any of a variety of bus architectures. The system memory includes read only memory (ROM) 208 and random access memory (RAM) 210. A basic input/output system 212 (BIOS), which contains basic routines that help transfer information between elements within the computing system 104, is stored in ROM 208.

The computing system 104 further includes a hard disk drive 213 for reading from and writing to a hard disk, a magnetic disk drive 214 for reading from or writing to a removable magnetic disk 216, and an optical disk drive 218 for reading from or writing to a removable optical disk 219 such as a CD ROM, DVD, or other optical media. The hard disk drive 213, magnetic disk drive 214, and optical disk drive 218 are connected to the system bus 206 by a hard disk drive interface 220, a magnetic disk drive interface 222, and an optical drive interface 224, respectively. The drives and their associated computer-readable media provide nonvolatile storage of computer readable instructions, data structures, programs, and other data for the computing system 104.

Although the example environment described herein can employ a hard disk 213, a removable magnetic disk 216, and a removable optical disk 219, other types of computer-readable media capable of storing data can be used in the example system 104. Examples of these other types of computer-readable mediums that can be used in the example operating environment include magnetic cassettes, flash memory cards, digital video disks, Bernoulli cartridges, random access memories (RAMs), and read only memories (ROMs).

A number of program modules can be stored on the hard disk 213, magnetic disk 216, optical disk 219, ROM 208, or RAM 210, including an operating system 226, one or more application programs 228, other program modules 230, and program data 232.

A user can enter commands and information into the computing system 104 through input devices such as, for example, a keyboard 234, mouse 236, or other pointing device. These and other input devices are often connected to the processing unit 202 through a serial port interface 240 that is coupled to the system bus 206. Nevertheless, these input devices also can be connected by other interfaces, such as a parallel port, game port, or a universal serial bus (USB). An LCD display 242 or other type of display device is also connected to the system bus 206 via an interface, such as a video adapter 244.

The computer system 104 can operate in a networked environment using logical connections to one or more remote computers, such as a remote computer 246. The remote computer 246 can be a computer system, a server, a router, a network PC, a peer device or other common network node, and typically includes many or all of the elements described above relative to the computer system 104. The network connections include a local area network (LAN) 248 and a wide area network (WAN) 250. When used in a LAN networking environment, the computer system 104 is connected to the local network 248 through a network interface or adapter 252. When used in a WAN networking environment, the computing system 104 typically includes a modem 254 or other means for establishing communications over the wide area network 250, such as the Internet. In a networked environment, program modules depicted relative to the computing system 104, or portions thereof, can be stored in the remote memory storage device. It will be appreciated that the network connections shown are examples and other means of establishing a communications link between the computers can be used.

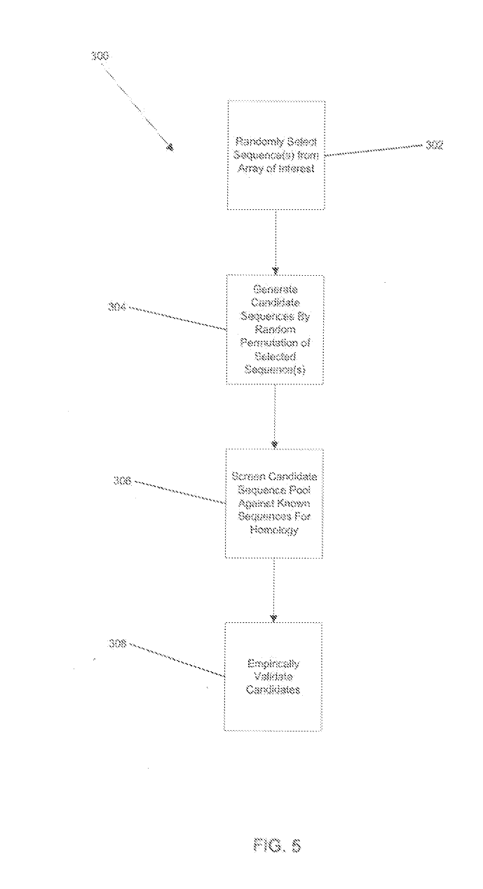

Referring now to FIG. 5, a flowchart 300 is provided illustrating operations that are performed in some embodiments. First, one or more biological probe sequences of interest are randomly selected from an array of interest 302. As used herein, the term “biological probe sequences” includes those sequences of a set of sequences that are designed to hybridize with target molecules (also referred to as biologically occurring molecules), such as nucleotide sequences, that can be present in a sample. Such sequences can be included on a chemical array. Next, a pool of candidate sequences is generated by randomly permuting the bases (or nucleotides) of each selected biological probe sequences 304. The term “permuting” as used herein shall mean to change the order or arrangement of bases within a sequence. One or more screening operations are then performed on the pool of candidate sequences. As an example of one screening operation, the candidate sequences are screened for similarity against known biological sequences of genome or transcriptome of the organism of interest to eliminate those having significant similarity with any known biological sequence 306. The best-alignment of a 60-mer negative control sequence to the human genomic sequence should contain no contiguous hits of more than 20 consecutive bases or about 33% of the probe sequence as determined by a BLAST search using default parameters. Using ProbeSpec with a index-seed size of 10 there should be hits with fewer than 20 mismatches across the length of the probe for the nearest hit in the genome.

The organism of interest is the organism, or any of the organisms, for which the array is designed to analyze samples from. Individual screening operations are performed by themselves or in addition to other screening operations. Then, in some embodiments, the remaining candidate sequences are empirically validated on a test array 308. For example, candidate sequences can be synthesized and then put on a test array (or synthesized in situ) and then the candidate sequences can be tested for hybridization with a test sample. Operations performed in some embodiments will now be discussed in greater detail.

Some embodiments include random selection of biological probe sequences from a set of sequences (e.g., such as a plurality of sequences designed for inclusion on a chemical array of interest). The array of interest is the particular array for which negative control probes are being designed. The selected biological probe sequences then serve as the starting point from which candidate probe sequences are generated (as further described below). In some embodiments, when biological probe sequences are used as the starting point, the resulting candidate probe sequences will match the base composition (e.g., A/T/G/C %) of the biological probe sequences in the array of interest. In some embodiments, the resulting candidate probes can be used to more accurately measure both residual spatially varying background as well as the sequence specific background variations. In some embodiments, by randomly choosing the biological probes to use for generating the candidate probes, the resulting negative control probe sequences have base compositions and thermodynamic properties that closely represent those distributions for the biological probes themselves.

In some embodiments, screening can include screening the candidate sequences for base composition properties such as for A/C/T/G content, the presence or absence of homopolymeric runs, screening for hairpin loops or for thermodynamic characteristics such as for melting temperature. In general, each screening operation reduces the pool of potential candidate sequences. Methods of screening according to such characteristics are described in U.S. patent application Ser. No. 11/232,817, filed Sep. 21, 2005, incorporated by reference herein.

Arrays can include any desired number of biological probe sequences. By way of example, arrays can include 10s, 100s, 1,000s, or 10,000s of different biological probe sequences. Any desired number of the biological probe sequences can be randomly selected. The desired number can depend on the number of biological probe sequences in the array of interest. In some embodiments, the number of biological probe sequences selected is equal to between about 0.1% and 20% of the biological probe sequences on the array of interest.

It will be appreciated there are many ways of randomly selecting individuals from among a group. By way of example, different biological probe sequences can be assigned different reference numbers and then a subset of the reference numbers can be randomly or pseudo-randomly selected. The term “random” as used herein shall include pseudo-random unless indicated to the contrary. Techniques of random number selection can include lottery methods, the use of random number tables, entropy approaches, and the like. It will also be appreciated that there are many ways of using computer systems to automatically generate random numbers. Further, techniques for generating random numbers can be implemented in many different programming languages. After random selection of biological probe sequences, the selected sequences are then used as the starting point for candidate probe generation.

In some embodiments, nucleotide base sequences are represented by the letters A/T/G/C. It will be appreciated that these letters correspond to the bases occurring in DNA (adenine, thymine, guanine, and cytosine). However, in some embodiments, other letters are used corresponding to components of other biopolymers, such as RNA or polypeptides. In addition, in some embodiments, letters are used corresponding to artificial components such as non-naturally occurring bases or peptides. As used herein the term “bases” or “monomer units” or “letters” can be used interchangeably though in specific contexts as will be apparent, the term “bases” or “monomer units” will refer to the chemical moieties, while “letters” will refer to a representation of the former.

Some embodiments include methods of generating candidate probe sequences. The term “candidate probe sequences” as used herein includes generated sequences that are later subject to one or more screening steps in order to produce negative control probe sequences. Biological probe sequences selected from an array of interest can serve as the starting point for the generation of a pool of candidate probe sequences. By way of example, the selected biological probe sequences can be randomly permuted to form a pool of candidate probe sequences. There are many techniques of random sequence permutation that can be used. By way of example, the letters (corresponding to bases) of a given selected biological probe sequence can be tallied with regard to the total number of each letter present. By way of example, assuming the selected biological probe sequences are 60 bases in length, a given selected biological probe sequence can be found to contain the following composition of bases: 13 A, 16 T, 15 G, and 16 C. A permuted random sequence can then be generated using this group of letters by randomly selecting one letter out of the group for each position in the permuted sequence until all of the 60 letters are used. In this case, the resulting permuted sequence would still contain a total 60 letters (specifically 13 A, 16 T, 15 G, and 16 C) but the sequence of letters would be different than the sequence of letters in the original selected biological probe sequence. It will be appreciated that there are many other techniques that can be used for generating random permuted sequences based on a given starting sequence.

The total number of possible unique random permutations depends on the total length of the sequence and the composition of different letters within the sequence. However, in the example of a sequence that is 60 bases in length having a relatively even distribution of bases, it will be appreciated that a very large number of random permutations are possible. It is estimated that only a fraction of these randomly generated permutation sequences are found within the sequences of all living organisms. An even smaller fraction would be found with the sequences of a given organism, such as the organism of interest. For any given length of random sequence generated, those that are found within the sequences of the organism of interest can be removed from the candidate pool through similarity screening, in silico, as described further below and/or by empirical testing (e.g., in a hybridization experiment).

In some embodiments, the pool of candidate sequences generated is screened for sequence similarity against the entire genome (for CGH arrays or arrays used for location analysis, e.g., ChIP-chip analysis) or the entire transcriptome (for expression arrays) of an organism from which samples to be tested will be obtained (organism of interest). The term “sequence similarity” as used herein shall refer to the degree to which two sequences are similar in their base sequence. Sequence similarity can be quantified in various ways known to those of skill in the art. Eliminating candidate sequences from the pool that have substantial similarity to sequences of an organism of interest helps to ensure that candidate sequences will be chosen that will function as negative controls. Similarity screening can be performed using many different tools available to those of skill in the art. A possible example includes determining similarity using the BLASTN program available at the website for the National Center for Biotechnology Information (NCBI). The BLASTN program uses the heuristic search algorithm BLAST (Basic Local Alignment Search Tool) to compare a nucleotide sequence (N) against a nucleotide sequence dataset. See Altschul et al., 1990, J. Mol. Biol., 215:403-10. The BLAST algorithm identifies regions of local similarity and then moves bi-directionally until the BLAST score declines. Another useful tool is BLAT. See Kent W J. BLAT-The BLAST-Like Alignment Tool. Genome Research, April 12(4):656-64. 2002. ProbeSpec is another useful tool that calculates the numbers of mismatches of nearest hits. See Doron Lipson, Peter Web, Zohar Yakhini (2002) “Designing Specific Oligonucleotide Probes for the Entire S. cerevisiae Transcriptome”, WABI '02, 17-21/9/02, Rome.

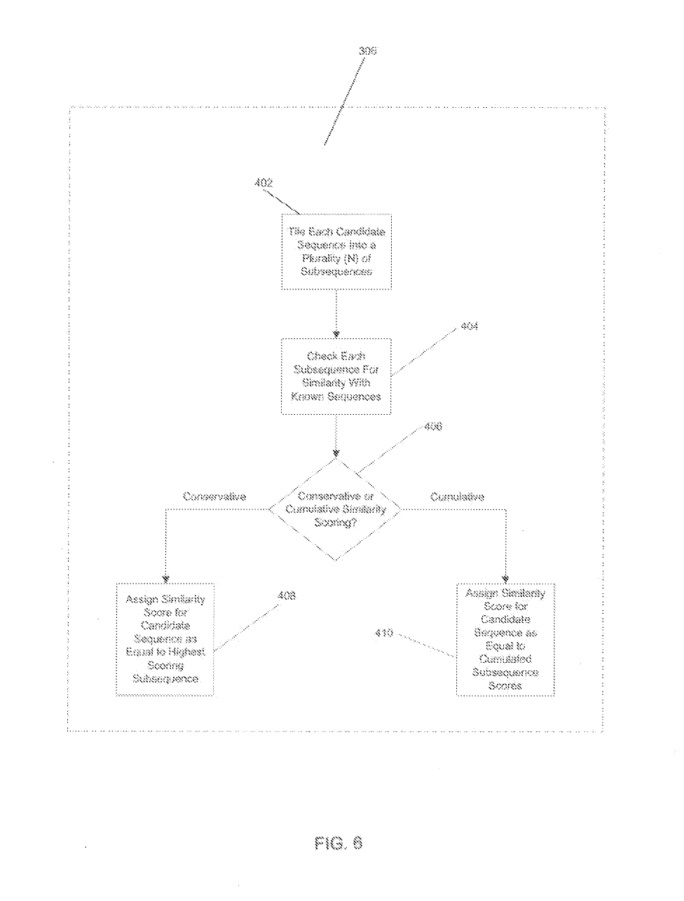

In some embodiments, subsequences of candidate sequences are screened for similarity against known biological sequences of an organism (or organisms) of interest. Referring now to FIG. 6, in some embodiments, a given candidate sequence can be subdivided into a plurality of overlapping or non-overlapping subsequences 402, each of which is then screened for similarity against known biological sequences of an organism of interest 404. For example, a candidate sequence having a length of 60 bases could be subdivided into three distinct subsequences wherein the first subsequence comprises bases 1-30 of the candidate sequence, the second subsequence comprises bases 15-45 of the candidate sequence, and the third subsequence comprises bases 30-60 of the candidate sequence. Then each of these subsequences can be compared with a database of known sequences to check for significant similarity 404. It is believed that screening subsequences can offer advantages in that it can make it less likely that any sub-region within a given candidate sequence has a significant match from within the genome or transcriptome of the organism of interest. However, in some embodiments similarity screening is performed using the full candidate sequences.

Similarity can be scored in various ways. In some embodiments, histograms showing the closest matches found are prepared for each sequence or subsequences. Specifically, a histogram is generated showing the number of hits as a function of “distance” of candidate sequences or subsequences from known sequences within the genome or transcriptome of the organism of interest. For example, a distance of 0 base pair(s) corresponds to a candidate sequence that has a direct match in the known sequences within the genome or transcriptome of the organism of interest. Similarly, a distance of 1 base pair(s) corresponds to a candidate sequence having a match in the known sequences within the genome or transcriptome of the organism of interest that is different by only 1 base. Then a score is assigned based on the histogram with “smaller distance” hits (more similar) increasing the score more than “longer distance” hits (less similar). For example, each hit with a distance of 1 base pair might result in increasing the total score for the candidate sequence by 15 units whereas each hit with a distance of 2 base pairs might result in increasing the total score for the candidate sequence by only 12 units. This is only one example of how similarity can be scored. It will be appreciated that scoring can be conducted in many different ways as desired.

In the example of similarity screening performed on subsequences after subdividing the candidate sequences, scoring can be tallied in either a conservative or cumulative manner (see decision 406 in FIG. 6). In some embodiments of the conservative approach 408, scoring can be done by calculating the distribution of similarity scores for each of the subdivided subsequences from a given candidate sequence. Then, the subsequence having the highest similarity score to any sequence from the organism of interest is used to set the score for the overall candidate sequence from which the subsequences are taken. For example, if there are three subsequences in a given candidate sequence and one of the sequences has a score that is higher than the other two, then that higher score is taken as the score for the whole candidate sequence.

Alternatively, similarity scoring for candidate sequences can be done in a cumulative manner. In some embodiments of the cumulative approach 410, the similarity scores for each subsequence are calculated and then cumulated or averaged. For example, assuming there are 3 subsequences for a given candidate sequence and each subsequence produces similarity scores of X, Y, and Z respectively, then the similarity score for the given candidate sequence can be set as either the sum of X, Y, and Z or the average of X, Y, and Z. While some specific examples of calculating similarity scores for candidate sequences have been illustrated herein, it will be appreciated that there are many other ways of calculating similarity scores.

After similarity scores are calculated for candidate sequences, those sequences resulting in scores that indicate significant similarity with one or more naturally occurring sequences in the genome or transcriptome of the organism of interest are removed from the candidate sequence pool. The precise cut-off level for similarity scores will depend on various factors including the length of the candidate sequences, the stringency of wash steps used in the hybridization protocol for the array of interest, scoring method, etc.

Candidate probe sequences that have significant similarity to naturally occurring sequences are undesirable for use as negative controls. In some embodiments, a BLAST raw score (S) is used to select those sequences that do not have significant similarity to known biological sequences. It will be appreciated that BLAST raw score thresholds can be set as desired. In some embodiments, candidate negative control sequences producing any matches against biological sequences with a BLAST raw score of greater than or equal to about 20 are not used. In some embodiments, candidate negative control sequences producing any matches against biological sequences with a BLAST raw score of greater than or equal to about 25 are not used. In some embodiments, candidate negative control sequences producing any matches against biological sequences with a BLAST raw score of greater than or equal to about 30 are not used. In some embodiments, candidate negative control sequences producing any matches against biological sequences with a BLAST raw score of greater than or equal to about 30.23 are not used.

In some embodiments, candidate sequences predicted to form a hybrid with any naturally occurring sequence in the genome or transcriptome of the organism of interest having a predicted Tₘ sufficiently high that the hybrid would be predicted not to melt off during the most stringent post-hybridization was step used in the hybridization protocol are removed from the candidate sequence pool. In some embodiments, candidate sequences having sequence identity of greater than 10 contiguous complementary base pairs, or equally stable longer homologous sequences containing deletions or mismatches, are removed from the candidate sequence pool. In some embodiments, candidate sequences having sequence identity of greater than 15 contiguous complementary base pairs, or equally stable longer homologous sequences containing deletions or mismatches, are removed from the candidate sequence pool.

Closely related to similarity screening, some embodiments can include screening candidate probes for hybridization potential. Hybridization potentials can be calculated using various algorithms known to those of skill in the art. By way of example, hybridization potentials for given sequences can be calculated using a program available online at The Bioinformatics Center at Rensselaer and Wadsworth website (bioinfo.rpi.edu).

One manner of expressing hybridization potential is as ΔG (change in Gibbs free energy) in units of kcals/mol. In some embodiments, candidate sequences having hybridization potential with any naturally occurring biological sequence of a magnitude greater than or equal to −5 kcal/mol are discarded. In some embodiments, candidate sequences having hybridization potential with any naturally occurring biological sequence of a magnitude greater than or equal to −10 kcal/mol are discarded. In some embodiments, candidate sequences having hybridization potential with any naturally occurring biological sequence of a magnitude greater than or equal to −15 kcal/mol are discarded.

In some embodiments, the selected biological probes from the array of interest and/or the pool of candidate probes are screened by their predicted melting temperature with their respective hypothetical complements. In the denaturation of DNA, melting temperature is taken as the midpoint of the helix-to-coil transition. It will be appreciated that there are many different algorithms known to those of skill in the art that allow the prediction of melting temperature based on primary structure (the sequence itself. Examples of such algorithms include that described in Dimitrov and Zuker, 2004, Biophysical Journal, 87:215-226. The higher the melting temperature, the more energetically stable the duplex or hybridization is.

In some embodiments, candidate sequences having a predicted melting temperature outside the range of about 75° C. to about 85° C., assuming molecule concentrations of between about 1×10⁻⁸ M and 1×10⁻¹⁰ M, are discarded. In some embodiments, candidate sequences having a predicted melting temperature outside the range of about 78° C. to about 82° C., assuming molecule concentrations of between about 1×10⁻⁸ M and 1×10⁻¹⁰ M, are discarded. In some embodiments, candidate sequences having a predicted melting temperature outside the range of about 79.5° C. to about 80.5° C., assuming molecule concentrations of between about 1×10⁻⁸ M and 1×10⁻¹⁰ M, are discarded.

Thermodynamic properties related to the formation of stable structures, such as hairpins, can be calculated in an analogous manner to those of duplex formation. This information can similarly be used to reject candidate sequences if it is likely that the probe will exist in a hairpin formation in solution under the hybridization conditions.

Some embodiments include screening techniques that rely on dataset(s) containing known biological sequences from the organism of interest. Some arrays are designed for use with samples taken from specific organisms. The specific organism(s) that a given array is designed to test samples from is the “organism(s) of interest”. Many projects being conducted by those of skill in the art continue to add to the total pool of known biological sequences for many different organisms. The dataset used for similarity screening can be drawn from one or more databases.

Exemplary databases containing known biological sequences include the NCBI nt database (ncbi.nih.gov), the TIGR (The Institute for Genomic Research) gene indices (tigr.org/tdb/tgi/index.shtml), and the NCBI's Unigene datasets (ncbi.nlm.nih.gov/entrez/query.fcgi?db=unigene). In some embodiments, screening techniques are performed against one or more of the NCBI nt dataset, the TIGR gene indices, and the NCBI's Unigene unique datasets for H. sapiens, A thaliana, and C. elegans.

Those of skill in the art will appreciate that there are also other databases that are available and that contain additional sequences from many different organisms. Publicly available sequence databases include those maintained by: GenBank (Bethesda, Md. USA) (ncbi.nih.gov/genbank/), European. Molecular Biology Laboratory's European Bioinformatics Institute (EMBL-Bank in Hinxton, UK) (ebi.ac.uk/embi/), the DNA Data Bank of Japan (Mishima, Japan) (ddbj.nig.ac.jp/), the Ensembl project (ensembl.org/index.html), and The Institute for Genomic Research (TIGR) (tigr.org). Examples of databases that can be obtained and/or searched through the NCBI web portal (ncbi.nih.gov) include Entrez Nucleotides (including data from GenBank, RefSeq, and PDB), all divisions of GenBank, RefSeq (nucleotides), dbEST, dbGSS, dbMHC, dbSNP, dbSTS, TPA, UniSTS, PopSet, UniVec, WGS, Entrez Protein (including data from SwissProt, PIR, PRF, PDB, and translations from annotated coding regions in GenBank and RefSeq), RefSeq (proteins), and many others.

It will be appreciated that some datasets are directed to certain types of sequence information. By way of example, some datasets are directed to genomic sequences, while other datasets are directed to expressed sequences. Still other datasets are directed to polypeptide sequences. The appropriate dataset for use will depend on both the type of array intended (CGH, expression, etc.) and the identity of the organism of interest.

Some embodiments include using a computer system to screen candidate sequences against databases of known sequences. Many available sequence databases can be accessed with computer programs in a way that facilitates automated screening of candidate sequences. Some embodiments include a computer program that automatically screens candidate sequences against databases of known sequences.

Some embodiments include empirically validating candidate sequences. Candidate sequences can be empirically validated by putting the sequences on a test array and then testing hybridization of a sample with sequences on the test array. In the example of CGH arrays, the test sample used for validation testing can simply include any type of DNA containing sample from the organism since any normal diploid cell line contains the entire genome of the organism. In the example of expression arrays, since no single RNA sample includes all targets that can be expressed in any cell, the test sample will frequently represent a variety of tissue types or tissue conditions. By way of example, the test sample can include a mixed tissue sample (such as Universal Reference RNA, available from Stratagene, La Jolla, Calif.), a highly expressive cell line (such as HeLa), and/or a collection of tissues including unusual tissue types such as stressed cells, fetal tissue, and the like. In some embodiments, candidate sequence testing includes demonstrating that DNA and/or RNA from the test sample does not hybridize to the control probes under conditions (e.g., temperature, salt concentrations, sample concentrations, etc.) similar to that expected to be used in the hybridization protocol for the array of interest.

With arrays that are read by detecting fluorescence, the substrate can be of a material that emits low fluorescence upon illumination with the excitation light. Additionally, the substrate can be relatively transparent to reduce the absorption of the incident illuminating laser light and subsequent heating if the focused laser beam travels too slowly over a region.

In some embodiments, the disclosure provides methods for screening candidate probe sequences, in order to obtain candidates for use as negative control probes, comprising: selecting a subset of probe sequences from a set of sequences randomly; generating a plurality of candidate probe sequences by randomly permuting the selected probe sequence; and screening the candidate probe sequences for sequence similarity to biologically occurring sequences. In some embodiments, the method further comprises selecting a negative probe sequence from the candidate probe sequences wherein the negative probe sequence does not have significant sequence similarity to the biologically occurring sequences. Probe sequences can additionally be screened based on melting temperature (Tm). In some embodiments, the method comprises discarding candidate sequences having a melting temperate (Tm) outside the range of about 78° C. to about 82° C.

In some embodiments, one or more steps of the method can be performed using a computer.

In some embodiments, the biologically occurring sequences comprise at least 50%, at least 90% or the entire genome of a biological organism, for example, the genome of a mammal such as a human being. In some embodiments, the biologically occurring sequences comprise at least 50%, at least 90% or the entire transcriptome of a biological organism, for example, the transcriptome of a mammal such as a human being. In some embodiments, screening the candidate probe sequences for sequence similarity to biologically occurring sequences comprises screening a set of candidate probe sequences against a database of known sequences. In some embodiments, the set of sequences includes sequences complementary to nucleic acid sequences from an organism of interest, and the database comprises sequences from the organism of interest.

In some embodiments, screening the candidate probe sequences for sequence similarity to biologically occurring sequences comprises subdividing each candidate probe sequence into a plurality of corresponding candidate probe subsequences. The method can further comprise scoring the sequence similarity of each candidate probe sequence according to the sequence similarity of the corresponding candidate probe subsequences.

Methods according to some embodiments of the disclosure can further comprise generating a database of negative probe sequences. As discussed above, in some embodiments, a negative probe sequence does not have significant sequence similarity to biologically occurring sequences, such as for example, the genomic sequences of an organism (e.g., a mammal, such as a human being). In some embodiments, the genomic sequences comprise at least about 50%, at least 90% or 100% of the genomic sequences of an organism, such as a mammal (e.g., a human being). In some embodiments, the biologically occurring sequences comprise the sequences of a transcriptome and in some embodiments, at least 50%, at least 90%, or 100% of the transcriptome of a mammal, such as a human being.

In some embodiments, the methods comprise receiving sequence information for a negative probe sequence and synthesizing the negative probe sequence. Probe sequences can be synthesized by a variety of methods, including, but not limited to in situ synthesis on a solid support (e.g., an array substrate).

The methods can further include empirically testing candidate probe sequences by contacting the probe sequences to a test sample of target sequences and monitoring binding of the probe sequences to the target sequences. For example, candidate probe sequences can be included on an array substrate which can then be contacted with target sequences. The array substrate can additionally include one or more test sequences designed to specifically hybridize to one or more sequences in a biological sample comprising the biologically occurring sequences. In some embodiments, the array substrate includes a positive control probe comprising a sequence known to be complementary to a sequence in the sample or a sequence which is spiked into the sample.

A negative probe sequence can be included in a probe set, which can be immobilized on an array, in some embodiments, for a variety of hybridization-based assays. For example, the probe sequence can be included on an array used in a CGH assay, a location analysis assay, a gene expression assay and the like. Optionally, the probe can be empirically validated as described above before inclusion in the probe set. In some embodiments, a negative probe sequence comprises a sequence selected from SEQ ID NOS: 1-44. Some embodiments of the disclosure include a probe set comprising at least two nucleic acid molecules comprising sequences selected from the group consisting of SEQ ID NOS: 1-44 and an array comprising one or more probe sequences selected from the group consisting of SEQ ID NOS: 1-44.

In some embodiments, methods according to the disclosure further comprise synthesizing one or more negative control probe sequences. In some embodiments, a negative control probe sequence comprises a sequence length of 10 to 200 bases. In some embodiments, a negative control probe sequence comprises a sequence length of 60 bases. In some embodiments according to the disclosure, a probe includes a negative control sequence and a cleavable site for releasing the negative control probe from an array substrate on which it is immobilized. The probe can additionally or optionally include primer recognition sites for binding to a primer so that the probe can be copied in the presence of a primer, a polymerase and suitable reagents for performing a primer extension and/or amplification reaction.

In some embodiments, the disclosure further provides a probe sequence comprising a negative control probe sequence and a biological probe sequence (i.e., a sequence designed to specifically hybridize to a biologically occurring sequence) for detecting a target sequence in a sample. In some embodiments, the negative control probe sequence is proximal to a solid support on which the probe is immobilized, to link the biological probe sequence to the solid support (either directly or via an additional chemical moiety to which the negative control probe sequence is attached). In some embodiments, an additional parameter used to screen the negative control probe sequence is an absence of secondary structure or ability to form hairpins, such that the negative control probe sequence has minimal likelihood of forming secondary structure. In some embodiments, the negative control probe sequence moves the biological probe sequence off the surface of the microarray and increases hybridization potential of the biological probe sequence (e.g., by reducing steric hindrance and increasing overall sequence accessibility).

In some embodiments, the disclosure provides an array comprising at least one probe comprising a negative control probe sequence and a biological probe sequence. In still some embodiments, the array comprises a plurality of probes comprising a negative control probe sequence and a biological probe sequence. Within the plurality, the negative control probe sequences can be the same or different in some embodiments, though in some embodiments, they are the same. Similarly, within the plurality the biological probe sequence can be the same or different, though in some embodiments, the biological probe sequences are different. In some embodiments, the plurality can comprise the same negative control probe sequences and different biological probe sequences.

In some embodiments, the disclosure also provides a computer readable medium having computer-executable instructions for performing steps of methods as described herein.

In some embodiments, the disclosure provides an apparatus for screening candidate probe sequences, the apparatus comprising: a memory store; and a programmable circuit in electrical communication with the memory store, the programmable circuit programmed to select probe sequences from a set of sequences randomly; generate a plurality of candidate probe sequences by randomly permuting the selected biological probe sequence; and to screen the candidate probe sequences for sequence similarity to biologically occurring sequences. The circuit can be further programmed to select a probe sequence from the candidate probe sequences that does not have significant sequence similarity to the biologically occurring sequences. The programmable circuit can be further programmed to screen candidate probe sequences other properties, such as melting temperature (Tm), for example. In some embodiments, the apparatus further comprises or communicates with a nucleic acid synthesis device, such as an inkjet printer for printing a nucleic acid array. In some embodiments, the nucleic acid synthesis device is responsive to the programmable circuit (e.g., directly or indirectly).

In some embodiments, the disclosure provides a system comprising a database of negative control probe sequences. In some embodiments, sets of negative control probe sequences are selected which correspond to sets of different biologically occurring sequences. A set includes a least one collection of nucleic acid sequences for a biological sample of interest—for example, the set can include human genomic sequences for a biological sample from a human being. In some embodiments, the set includes a plurality of different collections of biologically occurring sequences. For example, a set can comprise mouse genomic sequences and human genomic sequences, such that the database includes a set of negative control probes for a sample of mouse genomic sequences and a set of negative control probes for a sample of human genomic sequences. In some embodiments, the system further comprises a search engine for searching the database in response to an input identifying a set of biologically occurring sequences. For example, in some embodiments, in response to a user request for negative control probes for a sample of human genomic nucleic acids, the search engine will search the database to identify those negative control probe sequences that do not have significant similarity to any human genomic sequences.

In some embodiments, the system communicates with a user device comprising a display for displaying data relating to the negative probe sequences. The data can include but is not limited to: annotation data, sequence data, data relating to empirically determined hybridization properties of the probes, etc. In some embodiments, in response to a selection of one or more negative control probes (e.g., by selecting appropriate areas on a graphical user interface or display), a user can communicate an order for the one or more negative control probes to an entity that can provide the user with such probes (e.g., synthesized on an array or provided in a lyophilized form or in solution).

In some embodiments, the subject methods include a step of transmitting data or results from at least one of the detecting and deriving steps, also referred to herein as evaluating, as described above, to a remote location. By “remote location” is meant a location other than the location at which the array is present and hybridization occur. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart.

“Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

Kits

Also provided are kits for use in the subject methods, where in some embodiments such kits can comprise containers, each with one or more of the various reagents utilized in the methods, where such reagents include, but are not limited to, one or more of the following: a control nucleic acid construct as described herein; a single-stranded oligonucleotide comprising a reverse complement of a negative control probe; a double-stranded oligonucleotide comprising a reverse complement of a negative control probe; a nucleic acid vector (e.g., a cloning vector); a restriction endonuclease for use in inserting a double-stranded oligonucleotide into a vector; a collection of control nucleic acid constructs; at least two collections of control nucleic acid constructs; a host cell; a host cell transfected with a control nucleic acid construct; a transfection agent; PCR primers for amplifying a region of a control nucleic acid construct; labeling reagents, e.g., labeled nucleotides, and the like; a cross-linking reagent; an array of target nucleic acids; a hybridization solution. Where the kits are specifically designed for use in CGH applications, the kits can further include labeling reagents for making two or more collections of distinguishably labeled nucleic acids according to the subject methods. In some embodiments, reagents be prepared as a concentrated form (e.g., 10× concentrated) to be diluted upon use.

In some embodiments, a kit can further include instructions for using kit components in the subject methods. The instructions can be printed on a substrate, such as paper or plastic, etc. As such, the instructions can be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc., or can be obtained from the web.

### EXAMPLE 1

### Preparation of Genomic DNA

Comparative genomic hybridization (CGH) measures copy number variations at multiple loci simultaneously, allows for the detection of DNA sequence copy number aberrations throughout the genome as an important tool for studying cancer and developmental disorders and for the development of diagnostic and therapeutic targets.

Comparative genomic hybridization is a technique that allows for the detection of DNA sequence copy number aberrations throughout the genome. Microarrays containing 60mer oligonucleotide probes designed for CGH measurements provide a platform for detecting chromosomal alterations throughout a genome using high complexity total genomic DNA samples.

The highly processive DNA polymerase, phi29, is used to prepare aCGH templates from low mass DNA samples that yield high quality aCGH measurements, comparable to those derived from unamplified total genomic DNA.

The protocol for aCGH contains multiple steps for sample preparation and labeling. Such steps include phi29 based amplification of total genomic DNA, restriction enzyme digestion, and Cyanine-3/Cyanine-5 dye labeling.

50 ng genomic DNA was used as input for the phi29 amplification reactions. The phi29 amplified samples were digested with restriction enzymes, Cyanine-3 or Cyanine-5 labeled and hybridized to. CGH oligonucleotide microarrays. HCT116, a colon cancer cell line derived from a male (XY) patient, was selected for the experimental sample because its genomic aberrations are well characterized in the literature and it retains an intact copy of the X chromosome. Hybridizations with a female (XX) reference allows one to detect a single copy deletion of the X chromosome using phi29 amplified DNA from reactions with varying sample input, as well as monitor the ability to detect other known aberrations on the autosomes.

HCT116 (ATCC catalog #CCL-247) cells were grown in culture under conditions recommended by the supplier and genomic DNA was isolated using the DNeasy Tissue kit (Qiagen catalog #69504), as per the manufacturer's recommendations. Normal female (XX) DNA was purchased from Promega (catalog #G1521). Initial sample concentrations were verified using a NanoDrop™ ND-1000 Spectrophotometer (NanoDrop Technologies, Inc.). 1 μl of the sample (50 ng) was amplified using the RepliG phi29 amplification kit (Qiagen catalog #59045) according to the manufacturer's instructions. Duplicate amplifications were done for each of the XX sample and duplicate amplifications were done for the HCT116 sample. Duplicate amplification reactions in the absence of template DNA were performed in which 1 μl of DNA was replaced with H₂O.

45 μl of unpurified phi29 amplified material was digested using 50 Units of Alul (Promega catalog #R6281) and 50 Units of Rsal (Promega catalog #R6371) in a 100μl volume with 10 μl 10× Promega Buffer C. Digestions were carried out for 2 hours at 37° C. The digested samples were purified using QIAprep Spin Miniprep columns (Qiagen catalog #27106) and eluted as per the manufacturer's instructions. The samples were quantitated using a Nanodrop.™ Spectrophotometer, and all the samples had similar yields, ˜20-30 μg, including the no template amplification.

The Alul/Rsal digested genomic DNA samples were labeled using the BioPrime Array CGH Labeling kit (Invitrogen catalog #18095-012) according to the manufacturer's protocol, except 10 μg of amplified DNA was used in each reaction instead of 4 μg. Each XX amplification sample was labeled in duplicate using Cyanine-3-dUTP while the HCT116 cell line amplified DNA samples were labeled with Cyanine-5-dUTP. The two amplification reactions performed in the absence of DNA template were split and labeled with both Cyanine-3 and Cyanine-5. The appropriate experimental (Cyanine-5-HCT116) and reference (Cyanine-3-XX) samples were then combined and the Cyanine-3 and Cyanine-5 no template samples were also combined. The Cyanine-3/Cyanine-5 labeled samples were brought to 500μl with TE (10 mM Tris pH 8.0/1 mM EDTA) and purified using the Microcon YM-30 columns (Millipore catalog #42410). The 500μl samples were applied to the columns and centrifuged at 8000×g for 10 minutes. The flow through was discarded and an additional 450 μl TE was added to the sample on the column and centrifuged at 8000×g for 10 minutes. The column was inverted into a new 1.5 ml tube and centrifuged at 8000×g for 1 minute to elute the sample. Eluted samples were brought to a volume of 100 μl in H2O.

To the purified Cyanine-3/Cyanine-5 labeled samples the following hybridization blocking reagents were added: 50 μg Cot-1 DNA (Invitrogen #15279-011), 100 μg Yeast tRNA (Invitrogen #15401011), and 50 μl 10× Control Targets (Agilent catalog #5185-5976). The volume was brought to 250 μl with H2O and 250 μl 2× Hybridization Buffer (Agilent catalog #5185-5973) was added. The hybridization mixture was denatured at 100° C. for 1.5 minutes in a water bath. Samples were immediately transferred to a 37° C. water bath for 30 min. Samples were centrifuged for 5 minutes at 16,000×g and immediately applied to Agilent's Human Genome CGH Microarrays (catalog #G4410A) as per the manufacturer's recommendations. Hybridizations were performed at 65° C. for 17 hours. The microarrays were disassembled and washed according to Agilent's aCGH hybridization protocol (part # G4410-90010). Microarrays were immediately scanned in the Agilent DNA microarray scanner (catalog #G2565BA) using the default settings. Data was extracted using the Agilent Feature Extraction software 7.5.1 (catalog #G2567AA) using the default settings, except for the following modifications: 1) Background Subtraction, the average of negative control features was used and the spatial detrend option was turned off and 2) Dye normalization, only the linear option was selected.

### EXAMPLE 2

### Use of Control Nucleic Acid Constructs

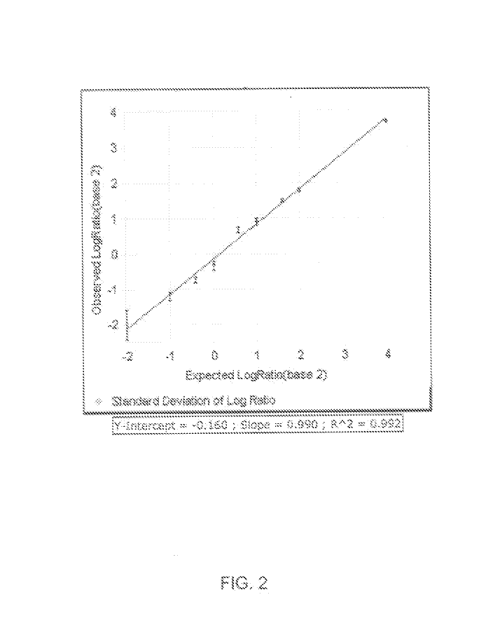

Control nucleic acid constructs were prepared for monitoring the microarray workflow process from sample digestion and labeling through hybridization in a aCGH assay. An Oligo aCGH Spike-in Kit was prepared and contained two control nucleic acid construct mixtures, A and B. Each mixture contains different control nucleic acid constructs designed to hybridize only with specific microarray probes without cross-hybridization to biological or other control probes on the microarray. The concentration of each control nucleic acid construct varies within a mixture and between each of the two mixtures. The Spike A and Spike B mixtures are added to the experimental and reference samples, respectively, in the restriction digestion step, prior to genomic DNA labeling. When the experimental and reference samples are co-hybridized on a microarray and feature extracted, the results of control nucleic acid constructs are captured in the QC report as a plot of expected versus observed log₂ ratios (FIG. 2). This plot can be used to monitor the system for linearity, sensitivity, and accuracy. In addition, the QC report displays coefficient of variation (CV) of replicates of negative control probes that are distributed across the microarray. Such measurements provide information on hybridization non-uniformities. The CGH microarray was designed to contain these specific probe sets that enable the use of control nucleic acid constructs. The Oligo aCGH Spike-in Kit is a tool for optimizing technique and for troubleshooting experiments. Concentrated Spike A and Spike B stocks are diluted with the Dilution Buffer provided in the kit. The diluted stocks are then spiked directly into the experimental and reference samples in the restriction enzyme digestion, prior to genomic DNA labeling. In this example, Spike mix A is added to the experimental sample and Spike mix B is added to the reference sample. The final relative amount of each of the 12 spike-in DNA transcripts is indicated in Table 2.

The Oligo aCGH Spike-in Kit contains:

### Spike A Mix, 14 μL

### Spike B Mix, 14 μL

### Dilution Buffer, 1.2 mL

The Oligo aCGH Spike-in Kit can be stored at −20° C. in a nondefrosting freezer for up to 1 year from the date of the receipt. The first dilutions of the Oligo aCGH Spike A and Oligo aCGH Spike B Mix can be stored up to 3 months in a non-defrosting freezer at −20° C. and freeze-thawed up to eight times.

TABLE 2
Final Relative Sample Amounts

| Control nucleic acid | Spike A Mix | Spike B Mix | Expected ratio |
|---|---|---|---|
| construct name | relative mass | relative mass | amounts (A/B) |
| SM_01 | 0 | 2 | 0:2 |
| SM_02 | 0.5 | 2 | 0.5:2 |
| SM_03 | 1 | 2 | 1:2 |
| SM_04 | 2 | 1 | 2:1 |
| SM_05 | 1.5 | 2 | 1.5:2 |
| SM_06 | 2 | 2 | 2:2 |
| SM_07 | 6 | 6 | 6:6 |
| SM_08 | 3 | 2 | 3:2 |
| SM_09 | 4 | 2 | 4:2 |
| SM_10 | 6 | 2 | 6:2 |
| SM_11 | 8 | 2 | 8:2 |
| SM_12 | 32 | 2 | 32:2 |

The nucleic acid constructs in Table 2 were prepared by PCR amplification (using SEQ ID NO:45 and SEQ ID NO:46 as PCR primers) of 12 different lambda vector constructs each of which contained a unique ˜60 bp insert (SEQ ID NOS:1-12, respectively) into the EcoR1 site.

Before using the Spike A Mix and Spike B Mix, the stock solutions are vortexed vigorously and briefly centrifuged to spin contents to the bottom of the tube prior to opening. Spike A Mix and Spike B Mix were diluted prior to use. The concentration of the spike mixes added to the experimental and reference samples is a function of the sample DNA mass. Table 3 indicates the appropriate dilutions of the Spike A Mix and Spike B Mix for the various starting amount of genomic DNA. Regardless of which labeling dye is to be used, Spike A Mix is always added to the experimental sample and Spike B Mix is always added to the reference sample.

### Reagents:

### Genomic DNA Labeling Kit PLUS (50) Agilent p/n 5188-5309

### Oligo aCGH Hybridization Kit (25) Agilent p/n 5188-5220

### Oligo aCGH Wash Buffer 1, 4 L Agilent p/n 5188-5221

### Oligo aCGH Wash Buffer 2, 4 L Agilent p/n 5188-5222

### Stabilization and Drying Solution Agilent p/n 5185-5979

TABLE 3
Dilutions of Spike A Mix and Spike B Mix (for 12 samples or less)

| | First Dilution | | Second Dilution | | |
|---|---|---|---|---|---|
| Starting | Spike A or | | Spike A or | | |
| amount | Spike B Mix | Dilution Buffer | Spike B Mix | Dilution Buffer | Final |
| of gDNA (ng) | volume (μL) | volume (μL) | volume (μL) | volume (μL) | dilution |
| 200 | 2.0 | 18.0 | 2.0 | 28.0 | 1:150 |
| 300 | 2.0 | 18.0 | 3.0 | 27.0 | 1:100 |
| 400 | 2.0 | 18.0 | 4.0 | 26.0 | 1:75 |
| 500 | 2.0 | 18.0 | 5.0 | 25.0 | 1:60 |
| 600 | 2.0 | 98.0 | NA | NA | 1:50 |
| 700 | 2.0 | 83.7 | NA | NA | 1:42.9 |
| 800 | 2.0 | 73.0 | NA | NA | 1:37.5 |
| 900 | 2.0 | 64.7 | NA | NA | 1:33.3 |
| 1000 | 2.0 | 58.0 | NA | NA | 1:30 |
| 1100 | 2.0 | 52.5 | NA | NA | 1:27.3 |
| 1200 | 2.0 | 48.0 | NA | NA | 1:25 |
| 1300 | 2.0 | 44.2 | NA | NA | 1:23.1 |
| 1400 | 2.0 | 40.9 | NA | NA | 1:21.4 |
| 1500 | 2.0 | 38.0 | NA | NA | 1:20 |
| 1600 | 2.0 | 35.5 | NA | NA | 1:18.8 |
| 1700 | 2.0 | 33.3 | NA | NA | 1:17.6 |
| 1800 | 2.0 | 31.3 | NA | NA | 1:16.7 |
| 1900 | 2.0 | 29.6 | NA | NA | 1:15.8 |
| 2000 | 2.0 | 28.0 | NA | NA | 1:15 |
| 2100 | 3.0 | 39.9 | NA | NA | 1:14.3 |
| 2200 | 3.0 | 37.9 | NA | NA | 1:13.6 |
| 2300 | 3.0 | 36.1 | NA | NA | 1:13 |
| 2400 | 3.0 | 34.5 | NA | NA | 1:12.5 |
| 2500 | 3.0 | 33.0 | NA | NA | 1:12 |
| 2600 | 3.0 | 31.6 | NA | NA | 1:11.5 |
| 2700 | 3.0 | 30.3 | NA | NA | 1:11.1 |
| 2800 | 3.0 | 29.1 | NA | NA | 1:10.7 |
| 2900 | 3.0 | 28.0 | NA | NA | 1:10.3 |
| 3000 | 3.0 | 27.0 | NA | NA | 1:10 |

To prepare the Spike A Mix Final Dilution appropriate for 200 ng of gDNA starting experimental sample:

### 1 Perform a 1:10 dilution.

a Label a new, sterile 1.5 mL microcentrifuge tube “Spike A Mix First Dilution.”

b Pipette 18 μL of the dilution buffer, provided in the Spike-Mix kit, to the 1.5 mL microcentrifuge tube labeled “Spike A Mix First Dilution.”

c Add 2 μL of the concentrated Spike A Mix to the 18 μL of the dilution buffer.

d Mix well and briefly centrifuge.

### 2 Perform a 1:15 dilution of the first dilution.

a Label a new, sterile 1.5 mL microcentrifuge tube “Spike A Mix Final Dilution.”

b Pipette 28 μL of the dilution buffer, provided in the Spike-Mix kit, to the 1.5 mL microcentrifuge tube labeled “Spike A Mix Final Dilution.”

c Pipette 2 μL from the tube labeled “Spike A Mix First Dilution” and add it to the 28 μL of dilution buffer in the tube labeled “Spike A Mix Final Dilution.”

d Mix well and briefly centrifuge.

### To prepare the Spike A Mix Final Dilution appropriate for

3 μg of gDNA starting experimental sample:

### 1 Perform a 1:10 dilution.

a Label a new sterile 1.5 mL microcentrifuge tube “Spike A Mix Final Dilution.”

b Pipette 27 μL of the dilution buffer, provided in the Spike-Mix kit, to the 1.5 mL microcentrifuge tube labeled “Spike A Mix Final Dilution.”

c Add 3 μL of the concentrated Spike A Mix to the 27 μL of the dilution buffer.

d Mix well and briefly centrifuge.

Sample data shown in FIG. 2 were obtained from an Oligo aCGH Spike-in Kit following the dilutions specified in Table 3.

### EXAMPLE 3

### Generation of Negative Control Sequences

While it will be appreciated that there are many different techniques for implementing embodiments as program code, this example provides a Matlab script as a specific example. The script takes biological probe sequences and creates random permutations of the sequences to generate a pool of random candidate sequences. The script then subdivides the candidate sequences into subsequences and checks for significant sequence similarity against a table containing known sequences from an organism of interest. The script then creates histograms for similarity scoring purposes.

```
      %MAKENEGATIVECONTROLPROBES (Matlab script)
      Multiplier=20;
      %Biological Probe Sequences:
      lod Sequences.mat
      for i=1:Multiplier
        %The scramble function randomly permutes the sequences:
        ScrambleSeqs=scramble(Sequences);
          if i==1
            Table60mers.Sequence=ScrambleSeqs;
      else
        Table60mers.Sequence=[Table60mers.Sequence;ScrambleSeqs];
              end
      end
      Table60mers.ProbeID=[1:length(Table60mers.Sequence)]’;
      Table60mers.Start=ones(size(Table60mers.ProbeID));
      %Tile 30-mer sub-probes through 60-mer probes at15-base intervals:
      Table30mers=subdivideprobes(Table60mers,30,15);
      Table30mers.ProbeID60mer=Table30mers.ProbeID;
      Table30mers.ProbeID=Table30mers.probeID*1000+Table30mers.Start;
      save WGA2_CandNegCont_Set2_Table30mers.mat Table30mers
      save WGA2_CandNegCont_Set2_Table60mers.mat Table60mers
      List30.ProbeID=Table30mers.ProbeID;
      List30.Sequence=Table30mers.Sequence;
      %export a text file that can be used by ProbeSpec for homology search of 30-mer test-
      sequences against human genome:
      table2tabtext(List30,‘WGA2_CandNegCont_Set2_Table30mers.lst’)
      %RUN PROBESPEC
      % load the resulting homology search file with a histogram of hits at various distances
      from 0–9 bases from the original 30-mer sequences:
      % load HomologyTable:
      load WGA2_CandNegCont_Set2_30mers_MAP.mat
      % load Table30mers:
      load WGA2_CandNegCont_Set2_Table30mers.mat
      % join Table30mers & HomologyTable on ProbeID:
      HomologyTable.ProbeID=double(HomologyTable.ProbeID)
      NewTable30mers=tablejoin(‘left’,Table30mers,HomologyTable,‘ProbeID’,‘=’, ‘ProbeID’)
      load WGA2_CandNegCont_Set2_Table60mers.mat
      % combine 30mer probes to make 60mer probes:
      % add histogram information for each triplet of 30-mer subsequences:
      HomologyTable60mers=combinesubseqhomologies(NewTable30mers,
      ‘ProbeID60mer’,‘Start’)
      NewTable60mers=tablejoin(‘left’,Table60mers,HomologyTable60mers,
      ‘ProbeID’,‘=’,‘UniFullSeqID’)
      % Score homologies for each probe, generate HomLogS2B score:
      [HomLogS2B,HomCat,NewTable60mers]=categorizehomology(NewTable60mers,1);
save NC_60mersHomologyTable.mat NewTable60mers
      % Keep only those probes with the best homology scores, HomLogS2B.
      figure,  %plot resulting homology score distribution:
      hist(Table.HomLogS2B,[floor(min(Table.HomLogS2B)):ceil(max(Table.HomLogS2B))])
```

The various embodiments described above are provided by way of illustration only and should not be construed to limit the claims. Those skilled in the art will readily recognize various modifications and changes that can be made without following the example embodiments and applications illustrated and described herein, and without departing from the true spirit and scope of the disclosure or the following claims.

1. A nucleic acid construct comprising:
a nucleic acid vector comprising an insert comprising a sequence complementary to a negative control sequence.

2. The nucleic acid construct of claim 1, wherein said insert is inserted into a restriction site of said vector.

3. The nucleic acid construct of claim 1, wherein the vector comprises a viral nucleic acid sequence.

4. The nucleic acid construct of claim 1, wherein the nucleic acid vector is selected from the group consisting of phage vectors and plasmids.

5. The nucleic acid construct of claim 2, wherein the vector comprises lambda phage gt11 and wherein said restriction site comprises an EcoR1 site.

6. The nucleic acid construct of claim 1, wherein said vector is double stranded.

7. The nucleic acid construct of claim 1, wherein said vector is linear.

8. An isolated nucleic acid molecule comprising lambda gt11 and an insert comprising a sequence selected from the group consisting of: SEQ ID NO:1, SEQ ID NO:2, SEQ ID NO:3, SEQ ID NO:4, and SEQ ID NO: 5.

9. An isolated nucleic acid molecule comprising an amplification product of the isolated nucleic acid molecule of claim 8 produced by PCR amplification using primers having the sequence of SEQ ID NO:45 and SEQ ID NO:46.

10. The nucleic acid construct of claim 1 wherein said insert occurs as a single copy in said vector.

11. The nucleic acid construct of claim 1 wherein said insert does not comprise a Tag sequence.

12. The nucleic acid construct of claim 1 wherein said insert does not comprise a concatenated sequence.

13. The nucleic acid construct of claim 1, wherein the length of said construct is in the range of 2 kilobases to 100 kilobases.

14. The nucleic acid construct of claim 1, wherein the length of said construct is in the range of 1 kilobase to 50 kilobases.

15. The nucleic acid construct of claim 9, wherein the length of said amplification product is about 2 kilobases.

16. The nucleic acid construct of claim 1 wherein said insert has a sequence length of 10 to 200 bases.

17. The nucleic acid construct of claim 1 wherein said insert has a sequence length of 60 bases.

18. A nucleic acid construct of claim 1 wherein said insert comprises DNA.

19. A composition comprising a pair of PCR primers capable of amplifying at least a portion of said insert of claim 1.

20. A transformed cell comprising the nucleic acid construct of claim 1.

21. A collection comprising a plurality of different nucleic acid constructs as described in claim 1, wherein each different control nucleic acid molecule in said collection has a different sequence.

22. A method for use in the preparation of a nucleic acid sample for microarray analysis, the method comprising the steps of: adding a nucleic acid construct to said sample, said nucleic acid construct comprising a nucleic acid vector having a single insert comprising a sequence complementary to a negative control sequence, and subjecting said sample to a plurality of processing steps.

23. The method of claim 22 wherein said processing steps comprise fragmenting said nucleic acid sample.

24. The method of claim 23 wherein said fragmenting comprises treatment with endonuclease.

25. The method of claim 23 wherein the method comprises a CGH assay.

26. The method of claim 23 wherein the method comprises a location analysis assay.

27. The method of claim 23 wherein the method comprises a gene expression assay.

28. The method of claim 23 wherein said processing steps comprise an immunoprecipitation step.

29. The method of claim 23 wherein said processing steps comprise a cross-linking step.

30. The method of claim 23 wherein said processing steps comprise an amplification step.

31. The method of claim 23 wherein said processing steps comprise a labeling step.

32. The method of claim 23 wherein the length of said construct is within about 10% to about 200% the length of nucleic acids in said sample.

33. The method of claim 23 wherein the length of said nucleic acid construct is within about 50% to about 150% the length of nucleic acids in said sample.

34. A composition comprising a control nucleic acid molecule comprising a sequence complementary to a negative control probe and an array comprising said negative control probe.

35. A method for monitoring hybridization of an eukaryotic nucleic acid sample to a nucleic acid array, said method comprising the steps of: (a) providing a nucleic acid array comprising a negative control probe and a plurality of nucleic acid test probes that specifically bind to eukaryotic nucleic acid targets; (b) providing a spiked sample comprising a eukaryotic nucleic acid sample and a nucleic acid construct, said nucleic acid construct comprising an insert that specifically binds to the negative control probe; (c) fragmenting said spiked sample; (d) contacting said spiked sample with said array; and (e) determining whether hybridization occurred between the negative control probe and the control nucleic acid molecule.

36. A kit comprising a nucleic acid construct and instructions for using the kit in a microarray hybridization assay, wherein the nucleic acid construct comprises:
a nucleic acid vector comprising an insert comprising a sequence complementary to a negative control sequence.

37. The kit of claim 36 wherein the nucleic acid construct comprises an isolated nucleic acid molecule comprising lambda gt11 and an insert comprising a sequence selected from the group consisting of: SEQ ID NO:1, SEQ ID NO:2, SEQ ID NO:3, SEQ ID NO:4, and SEQ ID NO:5.

38. The kit of claim 36 comprising a collection of different nucleic acid constructs, wherein each different nucleic acid construct in said collection comprises an insert having a unique sequence.

39. The kit of claim 38 comprising a first collection and a second collection of different nucleic acid constructs, wherein each insert in a collection has a unique sequence, wherein the sequences of the different nucleic acid constructs are same in the first and second collections, and wherein the ratios of the concentrations of the constructs in the first collection differs from the ratios of the concentrations of the constructs in the second collection.

40. The kit of claim 36 comprising an amplification product comprising the insert.

41. The kit of claim 36 comprising an array comprising a negative control probe.

42. The kit of claim 36 wherein the assay comprises a comparative genomic hybridization assay.

43. The kit of claim 36 wherein the assay comprises a location analysis assay.

44. The kit of claim 36 wherein the assay comprises a gene expression assay.

45. A method for preparing a control nucleic acid construct comprising the steps of:
a) providing a cloning vector, and
b) inserting into said vector a control nucleic acid molecule having a sequence complementary to a negative control sequence.

46. The method of claim 45 comprising transferring the product of step (b) into competent cells, and growing said cells.

47. The method of claim 46 comprising obtaining control nucleic acid construct from said cells.


SEQUENCE LISTING (44 sequences)

SEQ ID NO: 1 - DNA, 60 bp, human
gacttaaattcttcataactcgactacgagacctaatgtcggactaagttaaccaataaa

SEQ ID NO: 2 - DNA, 60 bp, human
tttattggttaacttagtccgacattaggtctcgtagtcgagttatgaagaatttaagtc

SEQ ID NO: 3 - DNA, 60 bp, human
tttgtaatctcgatacgcgtaagtttcgatcaggtaatttacatcgacatagacacccta

SEQ ID NO: 4 - DNA, 60 bp, human
tagggtgtctatgtcgatgtaaattacctgatcgaaacttacgcgtatcgagattacaaa

SEQ ID NO: 5 - DNA, 60 bp, human
cgataaaaagtcattgtatcgagtgataccgtaacctaccgttcgtagactattataaga

SEQ ID NO: 6 - DNA, 60 bp, human
tcttataatagtctacgaacggtaggttacggtatcactcgatacaatgactttttatcg

SEQ ID NO: 7 - DNA, 60 bp, human
tctcggtaaatagagtttcgtgcttatactagatgtagtctacgagatagacgctagatt

SEQ ID NO: 8 - DNA, 60 bp, human
aatctagcgtctatctcgtagactacatctagtataagcacgaaactctatttaccgaga

SEQ ID NO: 9 - DNA, 60 bp, human
aagtaacgtgagtagtatgatcatgttacgcgaggatcgttatcgagttacaataacata

SEQ ID NO: 10 - DNA, 60 bp, human
tatgttattgtaactcgataacgatcctcgcgtaacatgatcatactactcacgttactt

SEQ ID NO: 11 - DNA, 60 bp, human
tcgggtttacttgatatcaagcgcggttagaattgaatacgatgagacgaatttattaga

SEQ ID NO: 12 - DNA, 60 bp, human
tctaataaattcgtctcatcgtattcaattctaaccgcgcttgatatcaagtaaacccga

SEQ ID NO: 13 - DNA, 60 bp, human
atacgaatcttacgtagtttagtgtcgcttcactaaaaggctctatattcggatagtgca

SEQ ID NO: 14 - DNA, 60 bp, human
tgcactatccgaatatagagccttttagtgaagcgacactaaactacgtaagattcgtat

SEQ ID NO: 15 - DNA, 60 bp, human
ggctatcatagaaatgtagtcgaatcgtagcatactcgaattagatatctctatgctaag

SEQ ID NO: 16 - DNA, 60 bp, human
cttagcatagagatatctaattcgagtatgctacgattcgactacatttctatgatagcc

SEQ ID NO: 17 - DNA, 60 bp, human
caacgttgttatacgtcgttacctcaaaatgcgcgtaaaaacctgtgaactattataaag

SEQ ID NO: 18 - DNA, 60 bp, human
ctttataatagttcacaggtttttacgcgcattttgaggtaacgacgtataacaacgttg

SEQ ID NO: 19 - DNA, 60 bp, human
ttgaacttatgtaatctggtagtatcgagacaatcgttacagcgccatatgtaatgagaa

SEQ ID NO: 20 - DNA, 60 bp, human
ttctcattacatatggcgctgtaacgattgtctcgatactaccagattacataagttcaa

SEQ ID NO: 21 - DNA, 60 bp, human
tcgtgcagacttctacaacatcgagttctgcaacgtaataaccgtatgaataagactagt

SEQ ID NO: 22 - DNA, 60 bp, human
actagtcttattcatacggttattacgttgcagaactcgatgttgtagaagtctgcacga

SEQ ID NO: 23 - DNA, 60 bp, human
ctggtcttaatcgtcttgttaactaatacgggcatttacgagtcgatagacatataatca

SEQ ID NO: 24 - DNA, 60 bp, human
tgattatatgtctatcgactcgtaaatgcccgtattagttaacaagacgattaagaccag

SEQ ID NO: 25 - DNA, 60 bp, human
tgacaactagtttgcaatcgttataagtcgtattaacgcgaaattaacctgctaggaact

SEQ ID NO: 26 - DNA, 60 bp, human
agttcctagcaggttaatttcgcgttaatacgacttataacgattgcaaactagttgtca

SEQ ID NO: 27 - DNA, 60 bp, human
attagaactactataaatccggcgagattctatggcgcataacatgatagacagaacatt

SEQ ID NO: 28 - DNA, 60 bp, human
aatgttctgtctatcatgttatgcgccatagaatctcgccggatttatagtagttctaat

SEQ ID NO: 29 - DNA, 60 bp, human
gttaccgtttgaataataacggacggataaccctttgatacatcccaacgtataataagg

SEQ ID NO: 30 - DNA, 60 bp, human
ccttattatacgttgggatgtatcaaagggttatccgtccgttattattcaaacggtaac

SEQ ID NO: 31 - DNA, 60 bp, human
gtagagtatattgctttaatacgaccccgataagcacgatcgtattagacatagatgata

SEQ ID NO: 32 - DNA, 60 bp, human
tatcatctatgtctaatacgatcgtgcttatcggggtcgtattaaagcaatatactctac

SEQ ID NO: 33 - DNA, 60 bp, human
ataattcgttgactatagcacatttcgatcctcgttatgataccaatgaacggaagtctt

SEQ ID NO: 34 - DNA, 60 bp, human
aagacttccgttcattggtatcataacgaggatcgaaatgtgctatagtcaacgaattat

SEQ ID NO: 35 - DNA, 60 bp, human
cagatcgatcggtttatatgcgatttaacgccgctttcatcctaaagcgcaaattttaca

SEQ ID NO: 36 - DNA, 60 bp, human
tgtaaaatttgcgctttaggatgaaagcggcgttaaatcgcatataaaccgatcgatctg

SEQ ID NO: 37 - DNA, 60 bp, human
tacgtcaattcgtgatatgcctttcgattatcataccgaagagtcctttagtaagtttag

SEQ ID NO: 38 - DNA, 60 bp, human
ctaaacttactaaaggactcttcggtatgataatcgaaaggcatatcacgaattgacgta

SEQ ID NO: 39 - DNA, 60 bp, human
gaaactagtgaaacagagttcgctaagcgtctaaactcgagtttttacgaactaatacaa

SEQ ID NO: 40 - DNA, 60 bp, human
ttgtattagttcgtaaaaactcgagtttagacgcttagcgaactctgtttcactagtttc

SEQ ID NO: 41 - DNA, 60 bp, human
ggtattgttcttatattcatcgtgaccagtaaccaattgatatcggatttcggtttacag

SEQ ID NO: 42 - DNA, 60 bp, human
ctgtaaaccgaaatccgatatcaattggttactggtcacgatgaatataagaacaatacc

SEQ ID NO: 43 - DNA, 60 bp, human
ctatttctcgaaaccgttaaatcgaaatgttatgtccgctaatcgaaccactaatcgttt

SEQ ID NO: 44 - DNA, 60 bp, human
aaacgattagtggttcgattagcggacataacatttcgatttaacggtttcgagaaatag